(19) United States
(12) Patent Application Publication
(10) Pub. No.: US 2025/0076418 A1
(43) Pub. Date: Mar. 6, 2025

(54) CONTINUOUS OPERATION AND COMPENSATION OF A THREE-AXIS MAGNETOMETER

(71) Applicant: FieldLine Inc., Boulder, CO (US)

(72) Inventors: Ricardo Jimenez Martinez, Boulder, CO (US); Svenja Knappe, Boulder, CO (US); Kenneth J. Hughes, Lafayette, CO (US); Orang Alem, Erie, CO (US)

(21) Appl. No.: 18/824,092

(22) Filed: Sep. 4, 2024

Related U.S. Application Data
(60) Provisional application No. 63/580,888, filed on Sep. 6, 2023.

(51) Int. Cl. G01R 33/02; A61B 5/245; G01R 33/032

(52) U.S. Cl. CPC G01R 33/0206; A61B 5/245; G01R 33/032; A61B 2562/0223

(57) ABSTRACT

Various embodiments comprise a system to sense magnetic fields along x, y, and z measurement axes of a single-beam magnetometer. The x-axis is parallel to the propagation direction of the magnetometer light beam while the y and z axes are orthogonal to the light beam and to each other. The system comprises processing circuitry that processes the signal from the magnetometer to generate a z-axis control signal for a z-axis compensation coil. The processing circuitry processes the signal to generate a y-axis control signal for a y-axis compensation coil. The processing circuitry modifies y-axis current to drive the y-axis compensation coil based on the y-axis control signal and a modulation pattern. The processing circuitry delivers the modified y-axis current to the y-axis compensation coil to mitigate background magnetic field. The processing circuitry estimates magnetic field components along the x-axis based on the z-axis control signal and the modulation pattern.

### RELATED APPLICATIONS

This Patent Application claims the benefit of and priority to U.S. Provisional Patent Application 63/580,888 entitled, “CONTINUOUS OPERATION AND COMPENSATION OF A THREE-AXIS MAGNETOMETER” which was filed on Sep. 6, 2023, and which is hereby incorporated by reference in its entirety into this Patent Application.

### TECHNICAL FIELD

Various embodiments of the present technology relate to magnetometers and more specifically, to single beam magnetometers for three-axis magnetic field sensing.

### BACKGROUND

Magnetometer systems detect and characterize magnetic fields generated by a magnetic field source. The magnetometer systems measure the field strength and/or direction of the magnetic fields to characterize the sensed fields. Magnetometer systems may be used for anatomical magnetic imaging like Magnetoencephalography (MEG), Magnetocardiography (MCG), Magnetogastrography (MGG), Magnetomyography (MMG), and/or other types of anatomical magnetic field sensing operations. In anatomical magnetic imaging, the magnetometer systems measure the magnetic component of electromagnetic fields generated by neuronal activity within the body to map bodily functions. Exemplary magnetometers include atomic magnetometers, Optically Pumped Magnetometers (OPMs), gradiometers, nitrogen vacancy centers, Superconducting Quantum Interference Devices (SQUIDs), and the like.

Zero-Field Optically Pumped Magnetometers (ZF-OPMs) are a type of OPM with the capability to measure faint magnetic fields generated by the human body (e.g., the brain or heart). Due to their high sensitivity and potential for being wearable, ZF-OPMs are used to perform MEG, MCG, MGG, MMG, and other bio-magnetic sensing applications. ZF-OPMs typically have a limited dynamic range of a few nanoteslas. The limited range of ZF-OPMs requires precise control of the background magnetic field environment. Precise control is conventionally achieved using global magnetic shielding.

Global magnetic shielding uses a combination of a magnetically shielded enclosure and shim coils. The magnetically shielded enclosure comprises multiple layers of high-magnetic permeability material (e.g., mu-Metal) and one or two layers of copper and aluminum to attenuate background magnetic fields down to a few to tens of nanotesla. The shim coils are placed inside the enclosure and generate compensation fields that maintain the background magnetic field near zero in the vicinity of the ZF-OPMs. The conventional global shielding regimens are expensive and complex. The shielding enclosure typically only provides limited attenuation. The materials of the enclosure deform the magnetic fields applied by the shim coils and affect the spatial uniformity of the field. The lack of uniformity and limited attenuation reduces the range of motion of a patient wearing the ZF-OPMs.

An alternative approach to global field shielding controls the local field environment using electromagnetic coils within the ZF-OPMs. This approach has the potential to tolerate larger global magnetic fields and gradients, which could reduce complexity, cost of shielding enclosures, and eliminate shim coils. Local field shielding requires continuous and co-located measurements and compensation of all three vector components of the magnetic field at the position of the ZF-OPM. Conventional three-axis ZF-OPM sensing technologies use complex architectures based on dual-beams or multiple sensors and are not capable of reading all three magnetic field components at the same location. Other approaches capable of co-located measurements are not continuous.

Unfortunately, magnetometer systems do not efficiently shield magnetometers from background magnetic fields. Moreover, conventional three-axis ZF-OPMs are complex and do not effectively measure all three magnetic field components continuously at the same location.

### Overview

This Overview is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

Various embodiments of the present technology relate to solutions for magnetic field detection. Some embodiments comprise a method of operating a magnetic field detection system to sense magnetic fields along x, y, and z measurement axes of an intrinsically dual-axis single-beam magnetometer. The x-axis is parallel to the propagation direction of the light beam of the magnetometer while the y and z axes are orthogonal to the light beam and to each other. The method comprises processing a photodetector signal from the intrinsically dual-axis single-beam magnetometer to generate a z-axis control signal for a z-axis compensation coil oriented along the z-axis. The method further comprises processing the photodetector signal to generate a y-axis control signal for a y-axis compensation coil oriented along the y-axis. The method further comprises modifying the y-axis current to drive the y-axis compensation coil based on the y-axis control signal and a modulation pattern. The method further comprises delivering the modified y-axis current to the y-axis compensation coil to mitigate background magnetic field components along the y-axis. The method further comprises utilizing lock-in detection to estimate magnetic field components along the x-axis based on the z-axis control signal and the modulation pattern.

Some embodiments comprise a method of operating a magnetic field detection system to sense magnetic fields along x, y, and z measurement axes of an intrinsically single-axis single-beam magnetometer. The x-axis is parallel to the propagation direction of the light beam of the magnetometer while the y and z axes orthogonal to the light beam and to each other. The method comprises processing a photodetector signal from the intrinsically single-axis single-beam magnetometer to generate a z-axis control signal for a z-axis compensation coil oriented along the z-axis. The method further comprises modifying a y-axis current to drive a y-axis compensation coil oriented along the y-axis using a modulation pattern. The method further comprises delivering the modified y-axis current to the y-axis compensation coil to mitigate background magnetic components along the y-axis. The method further comprises modifying a current to drive an x-axis compensation coil oriented along the x-axis using the modulation pattern. The method further comprises delivering the modified x-axis current to the x-axis compensation coil to mitigate background magnetic field components along the x-axis. The method further comprises utilizing lock-in detection to estimate magnetic field components along the y-axis based on the z-axis control signal and the modulation pattern. The method further comprises utilizing lock-in detection to estimate magnetic field components along the x-axis based on the z-axis control signal and the modulation pattern.

Some embodiments comprise a magnetic field detection system to sense magnetic fields along x, y, and z measurement axes of an intrinsically dual-axis single-beam magnetometer. The x-axis is parallel to the propagation direction of the light beam of the magnetometer while the y and z axes orthogonal to the light beam and to each other. The system comprises processing circuitry. The processing circuitry processes a photodetector signal from the intrinsically dual-axis single-beam magnetometer to generate a z-axis control signal for a z-axis compensation coil oriented along the z-axis. The processing circuitry processes the photodetector signal to generate a y-axis control signal for a y-axis compensation coil oriented along the y-axis. The processing circuitry modifies a y-axis current to drive the y-axis compensation coil based on the y-axis control signal and a modulation pattern. The processing circuitry delivers the modified y-axis current to the y-axis compensation coil to mitigate background magnetic field components along the y-axis. The processing circuitry utilizes lock-in detection to estimate magnetic field components along the x-axis based on the z-axis control signal and the modulation pattern.

### DESCRIPTION OF THE DRAWINGS

Many aspects of the disclosure can be better understood with reference to the following drawings. The components in the drawings are not necessarily drawn to scale. Moreover, in the drawings, like reference numerals designate corresponding parts throughout the several views. While several embodiments are described in connection with these drawings, the disclosure is not limited to the embodiments disclosed herein. On the contrary, the intent is to cover all alternatives, modifications, and equivalents.

FIG. 1 illustrates an exemplary magnetic field detection system.

FIG. 2 further illustrates the exemplary magnetic field detection system.

FIG. 3 illustrates an exemplary operation of the magnetic field detection system.

FIG. 4 illustrates an exemplary operation of the magnetic field detection system.

FIG. 5 illustrates an exemplary Magnetoencephalography (MEG) system.

FIG. 6 illustrates an exemplary Optically Pumped Magnetometer (OPM).

FIG. 7 illustrates an exemplary MEG controller.

FIG. 8 further illustrates the exemplary MEG system.

FIG. 9 further illustrates the exemplary MEG system.

FIG. 10 further illustrates the exemplary MEG system.

FIG. 11 further illustrates the exemplary MEG system.

FIG. 12 illustrates an exemplary operation of the MEG system.

FIG. 13 illustrates an exemplary computing apparatus.

The drawings have not necessarily been drawn to scale. Similarly, some components or operations may not be separated into different blocks or combined into a single block for the purposes of discussion of some of the embodiments of the present technology. Moreover, while the technology is amendable to various modifications and alternative forms, specific embodiments have been shown by way of example in the drawings and are described in detail below. The intention, however, is not to limit the technology to the particular embodiments described. On the contrary, the technology is intended to cover all modifications, equivalents, and alternatives falling within the scope of the technology as defined by the appended claims.

### DETAILED DESCRIPTION

The following description and associated figures teach the best mode of the invention. For the purpose of teaching inventive principles, some conventional aspects of the best mode may be simplified or omitted. The following claims specify the scope of the invention. Note that some aspects of the best mode may not fall within the scope of the invention as specified by the claims. Thus, those skilled in the art will appreciate variations from the best mode that fall within the scope of the invention. Those skilled in the art will appreciate that the features described below can be combined in various ways to form multiple variations of the invention. As a result, the invention is not limited to the specific examples described below, but only by the claims and their equivalents.

The various embodiments described herein are directed to solutions for three-axes magnetic field detection using single beam magnetometers like Zero Field Optically Pumped Magnetometers (ZF-OPMs). In conventional approaches, a single-beam OPM approach capable of reading all three components of the local magnetic field is based on sequentially scanning a magnetic field in three orthogonal directions to detect zero-field magnetic resonances. While this approach is based on a simple OPM implementation, the detection range is limited to low frequency magnetic fields. Furthermore, the measurements are slow, and the approach is restricted to detecting a single component of the local field at any given time. Other conventional approaches rely on a single-beam OPM that uses three orthogonal modulating magnetic fields at frequencies below 200 Hz. The three vector components of the external field are then derived by demodulating the photodiode signal at the three modulating frequencies. While this approach enables continuous and simultaneous measurements of all three components of the local field, the sensitivity is poor and limited by low-frequency noise.

In other conventional approaches, complex OPMs (e.g., multi-laser) are used to measure the three vector components of the local magnetic field simultaneously. These approaches rely on the use of two independent optical beams and two independent photodetectors that are required to implement two independent dual-axis single-beam ZF-OPMs. While these methods enable continuous measurements in three orthogonal axes, one drawback is their complexity. For example, they require two optical beams, additional optics to route the beam, and two photodiodes to collect the transmitted beam. In some cases, two vapor cells are used, further increasing their complexity. Furthermore, these approaches do not measure the three vector components of the local magnetic field at the same location. For example, if the two beams overlap inside the vapor cell, the alkali spin polarization is adversely affected in the overlap region, and therefore for optimal performance the beam overlap volume must be reduced to the greatest extent possible. To minimize beam overlap, techniques may be employed including reducing the diameter of the beams to minimize beam overlap, and/or positioning or engineering the beams to create a small gap between the beams to further reduce beam overlap region inside the vapor cell. Therefore, it is desirable to achieve a simpler OPM architecture capable of reading all three components of the magnetic field continuously at the same location.

To overcome the above-described problems, a single beam ZF-OPM system is presented herein. The ZF-OPM system quickly reads and compensates all three components of a magnetic field at the same location and simultaneously. The ZF-OPM system relies on a simple single-beam sensor architecture and facilitates the use of simpler shielding enclosures. The ZF-OPM system may detect a wider magnetic field frequency range than conventional detection systems. The ZF-OPM system comprises improved sensitivity and noise resistance when compared to conventional OPM systems. Now referring to the Figures.

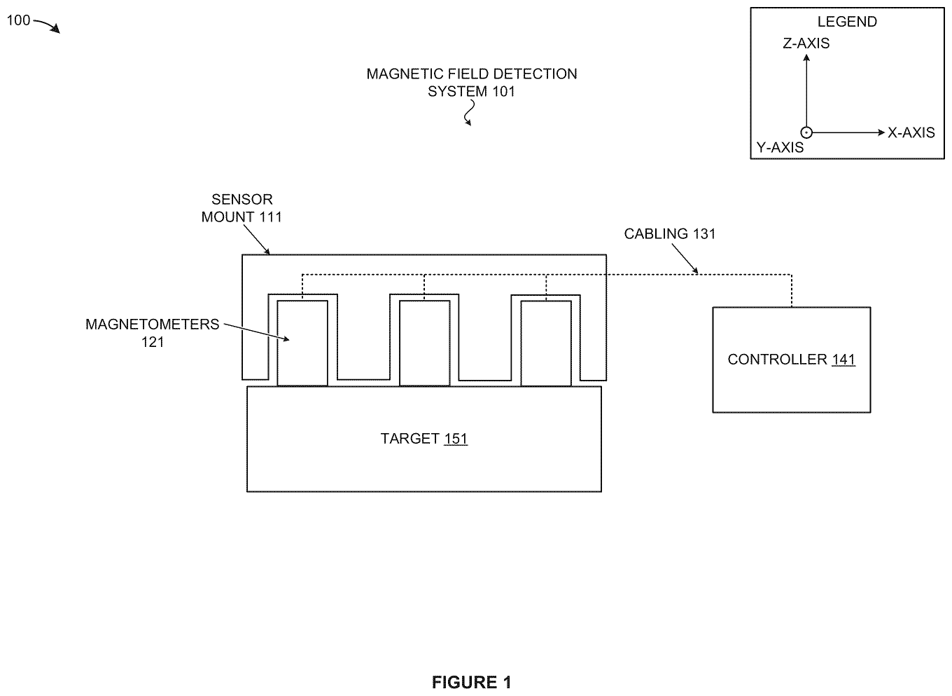

FIG. 1 comprises view 100. View 100 illustrates magnetic field detection system 101. Magnetic field detection system 101 performs operations like detecting magnetic fields and relating the detected magnetic fields to a magnetic field source. System 101 comprises sensor mount 111, magnetometers 121, cabling 131, controller 141, and target 151. Target 151 is representative of a magnetic field source. In other examples, system 101 may differ.

Various examples of magnetic field detection system operation and configuration are described herein. In some examples, target 151 generates a target magnetic field. Magnetometers 121 are positioned within the magnetic field generated by target 151. Controller 141 supplies current and control signaling to magnetometers 121 that drive magnetometers 121 to measure the target magnetic field. Magnetometers 121 are intrinsically sensitive to only two or one measurement axes. As illustrated in FIG. 1, the x-axis is parallel to the horizontal axis of the page, the z-axis is parallel to the vertical axis of the page, and the y-axis is parallel to a plane running through the page. For example, magnetometers 121 may be sensitive to components of the target magnetic field on the y and z axes, but may not be sensitive to components of the target magnetic field along the x-axis. Alternatively, magnetometers 121 may be sensitive to components of the target magnetic on the z-axis, but may not be sensitive to components of the target magnetic field on x and y-axes. To enable three axis sensitivity, controller 141 applies modulation patterns to the current supplied to magnetometers 121. Controller 141 may process the resulting signals received from magnetometers 121 to determine the components of the target magnetic field (e.g., strength, magnitude, direction, etc.) along all three measurement axes. Although illustrated as comprising three magnetometers, system 101 may comprise any number of magnetometers.

Sensor mount 111 comprises an apparatus to mount magnetometers 121. Mount 111 may comprise a rigid helmet, a flexible hat, a blanket, a sleeve, a vest, and the like. Typically, mount 111 is wearable by target 151. For example, if target 151 comprises an adult human, mount 111 may be shaped to fit over part of the human body (e.g., the head). Mount 111 may be constructed from plastic, carbon fiber, polymer, rubber, fabric, canvas, or other materials that provide structural support to mount 111 and that do not interfere in the sensing operations of magnetometers 121. As illustrated in FIG. 1, mount 111 comprises slots for magnetometers 121. Magnetometers 121 fit into the slots to connect to mount 111. Magnetometers 121 may connect to mount 111 using male/female sockets, ratchet mechanisms, set screws, springs, pistons, pneumatics, electronic actuators, and the like. The connectors between magnetometers 121 and mount 111 may be adjustable or static. For example, when mount 111 is worn by target 151, the connectors may propel magnetometers 121 through their respective slots until in contact with target 151 to contour magnetometers 121 to the surface geometry of target 151. Mount 111 may comprise embedded circuitry to communicatively couple magnetometers 121 and controller 141 via cabling 131. Alternatively, mount 111 may comprise conduit (or another type of passage) for cabling 131 to communicatively couple magnetometers 121 and controller 141.

Magnetometers 121 measure magnetic fields generated by target 151. For example, when target 151 comprises a human being, magnetometers 121 may sense magnetic fields generated in the brain, heart, muscles, and the like in target 151. Magnetometers 121 may be used to perform Magnetoencephalography (MEG), Magnetocardiography (MCG), Magnetogastrography (MGG), Magnetomyography (MMG), and/or other types of anatomical magnetic field sensing operations. It should be appreciated that target 151 may comprise any magnetic field source (e.g., the human brain), including non-biological field sources. Magnetometers 121 may comprise atomic magnetometers, OPMs, ZF-OPMs, gradiometers, nitrogen vacancy centers, and/or other types of optical based magnetic sensing devices. Magnetometer 121 may comprise subcomponents like atomic vapor cells, lasers, heaters, coils, photodetectors, processing circuitry, and communication circuitry.

Controller 141 comprises one or more computing devices that control the operation of magnetometers 121 to sense magnetic fields generated by target 151. Controller 141 is communicatively coupled to magnetometers 121 over cabling 131. The communication links may comprise metallic links, glass fibers, radio channels, or some other communication media. The links may use inter-processor communication, bus interfaces, Ethernet, WiFi, virtual switching, and/or some other communication protocol. For example, the communication links may be supported by sheathed metallic wires. Alternatively, the communication links may be supported by a wireless transceiver (e.g., antennas) to exchange signaling between controller 141 and magnetometers 121 over a wireless networking protocol like Bluetooth. Controller 141 may supply electric current over a wired connection to power magnetometers 121. Alternatively, magnetometers 121 may be battery powered.

The one or more computing devices of controller 141 comprise processors, memory, lock-in amplifiers, modulators, and transceivers that are connected over bus circuitry. The processors may comprise Central Processing Units (CPUs), Graphical Processing Units (GPUs), Digital Signal Processors (DSPs), Application Specific Integrated Circuits (ASICs), Field Programmable Gate Arrays (FPGAs), and the like. The lock-in amplifiers comprise demodulators and low-pass filters. The memory may comprise Random Access Memory (RAM), flash circuitry, Solid States Drives (SSDs), Hard Disk Drives (HDDs), Non-Volatile Memory Express (NVMe) SSDs, and the like. The memory stores software like operating systems, MEG applications, localization applications, Proportional Integral Derivative (PID) control applications, sensor data, and the like. The processors retrieve and execute the software from the memory to drive the operation of controller 141. In some examples, the lock-in amplifiers are implemented digitally by the processors (e.g., by the CPU).

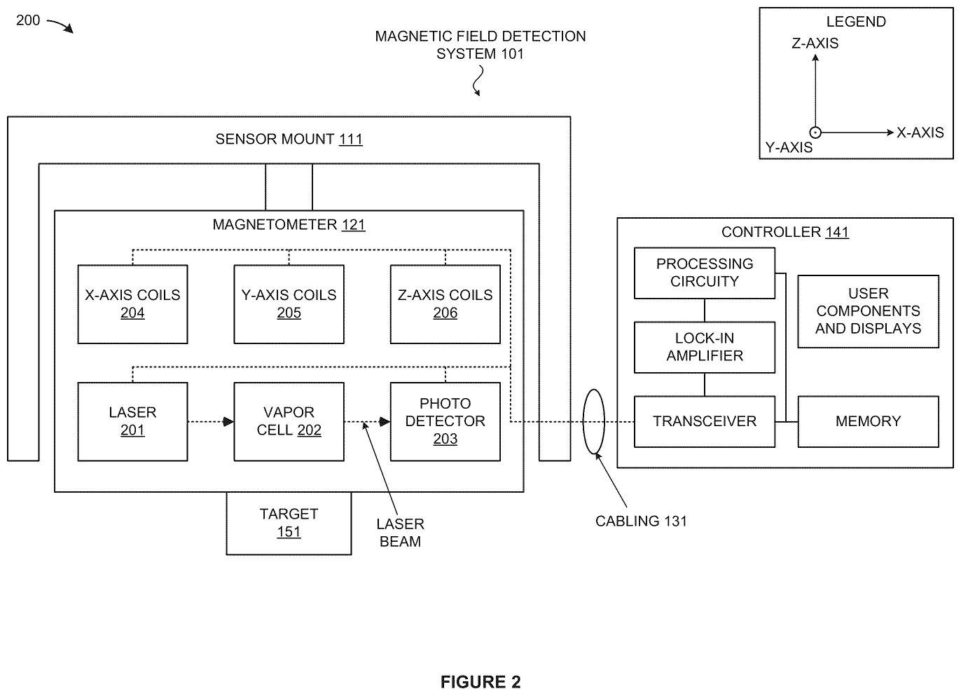

FIG. 2 comprises view 200. View 200 further illustrates magnetic field detection system 101. Magnetometers 121 are referred to in the singular for sake of clarity. In some examples, magnetometer 121 comprises laser 201, vapor cell 202, photodetector 203, x-axis coils 204, y-axis coils 205, and z-axis coils 206. Coils 204-206 are representative of circuitry to generate a magnetic field. Exemplary coil types include Helmholtz coils, saddle coils, solenoids, planar coils, metal wires, and the like. Magnetometer 121 typically comprises other components like transceiver circuitry, signal processors, flash circuitry, thermal packaging, heaters, collimating lenses, quarter wave plates, prisms, and/or other instruments and circuitry, however these additional components are omitted for clarity. Target 151 is magnetically linked to magnetometer 121. Magnetometer 121 is metallically linked to cabling 131 which is metallically linked to controller 141. Cabling 131 may be detachably coupled to controller 141 and/or to magnetometer 121.

Laser 201 comprises a light source to probe and polarize the vapor housed by vapor cell 202. The beam emitted by laser 201 passes through vapor cell 202 and is sensed by photodetector 203. Vapor cell 202 comprises an atomic device the encloses vapor. The vapor is selected for comprising properties relating to magnetic field sensitivity. For example, the vapor may comprise an alkali metal vapor. Photodetector 203 comprises a light sensor to absorb and measure the intensity of the beam emitted by laser 201. When the beam passes through vapor cell 202, a portion of the beam is absorbed by the vapor. The remaining portion of the beam exists cell 202 and is detected photodetector 203. The amount of light absorbed by the vapor correlates to the magnetic field present at the vapor cell. For example, the amount of absorbed light may indicate the magnitude and/or direction of a magnetic field at the location of the vapor cell.

Coils 204-206 comprise a set of coils arranged along the x, y, and z axes of vapor cell 202. As illustrated in FIG. 2, the x-axis runs along the horizontal plane of the page, the y-axis runs through the page, and the z-axis runs along the vertical axis of the page. The x-axis is parallel to the propagation direction of the beam emitted by laser 201. The y and z axes are orthogonal to the x-axis and to each other. Coils 204-206 emit a magnetic field to null or otherwise counteract background magnetic fields and/or to orient the sensing direction of magnetometer 121. X-axis coils 204 are oriented parallel to the propagation direction of the laser beam through vapor cell 202 (e.g., the x-axis) while y-axis coils 205 and z-axis coils 206 are orientated orthogonally to the propagation axis of the laser beam and to each other. X-axis coils 204 null background magnetic fields parallel to the propagation axis of the laser beam while coils 205 and 206 null background magnetic fields that are orthogonal to the beam. The background magnetic fields are representative of magnetic fields other than the target magnetic field generated by target 151. For example, the background magnetic field(s) may comprise the Earth's magnetic field or magnetic fields generated by magnets, electromagnets, electronic devices, electric current, and the like.

Controller 141 comprises transceiver circuitry (XCVR), memory, a processor, a lock-in amplifier, and user components and displays connected over bus circuitry. The processor comprises a CPU, GPU, DSP, FPGA, ASIC, and/or some other type of processing circuitry. The lock-in amplifier comprises a low-pass filter and demodulator. The memory comprises RAM, HDD, SSD, NVMe SSD, and the like. The processor retrieves the software from the memory and executes the software to drive the operation of system 101 as described herein. The processing circuitry of controller 141 controls magnetometer 121 to sense components of the magnetic field (e.g., magnetic fields generated by target 151 and/or background magnetic fields) at vapor cell 202 orthogonal to, and parallel with the propagation axis of the magnetometer light beam. In particular, the processing circuitry applies modulation patterns into the currents supplied to coils 204-206 to allow for magnetic field components at the location of vapor cell 202 to be derived along all three axes.

Advantageously, magnetic field detection system 101 efficiently shields magnetometers 121 from background magnetic fields. Moreover, magnetometers 121 effectively measure all three components of the magnetic field continuously and at the same location and have reduced complexity when compared to conventional three-axis ZF-OPMs.

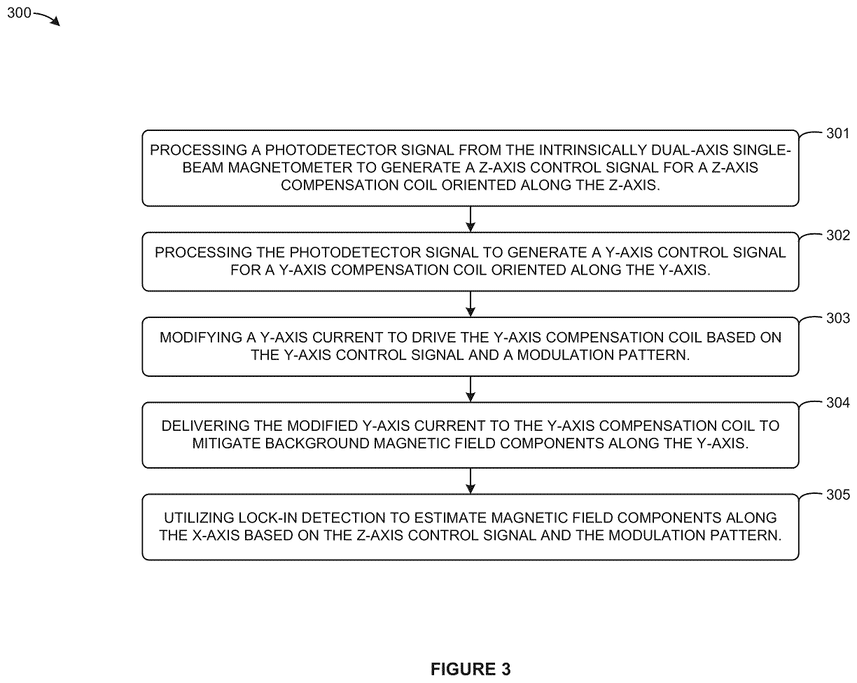

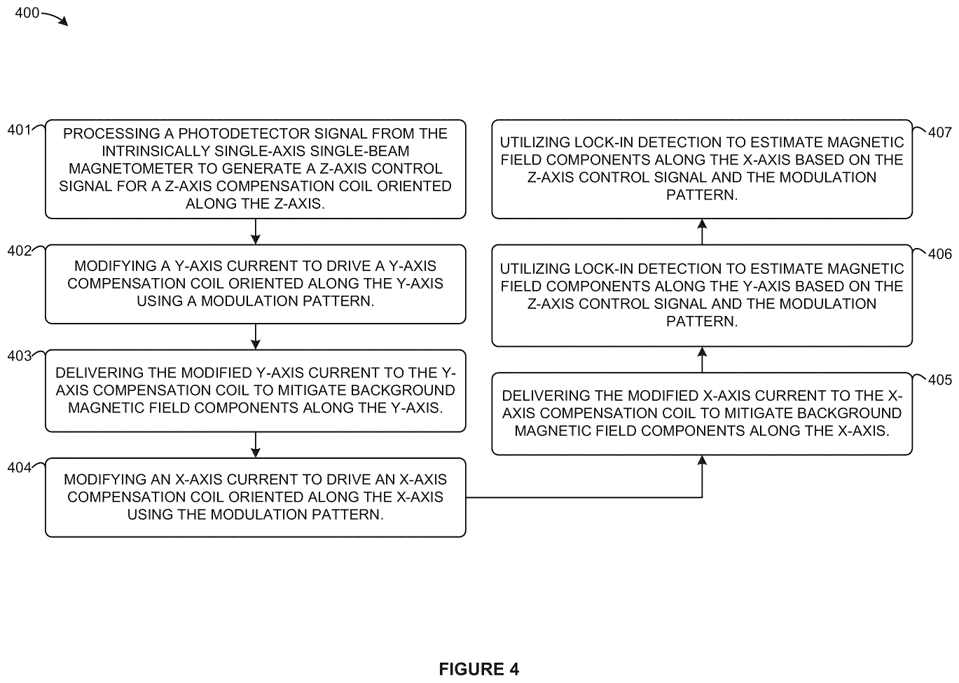

In some examples, magnetic field detection system 101 implements process 300 illustrated in FIG. 3. In some examples, magnetic field detection system 101 implements process 400 illustrated in FIG. 4. It should be appreciated that the structure and operation of system 101 may differ in other examples.

FIG. 3 illustrates process 300. Process 300 comprises an exemplary operation of magnetic field detection system 101 to sense magnetic fields along x, y, and z measurement axes of an intrinsically dual-axis single-beam magnetometer where the x-axis is parallel to the propagation direction of the magnetometer light beam while the y and z axes are orthogonal to the light beam and to each other. In other examples process 300 may differ. The operations of process 300 comprise processing the photodetector signal from the intrinsically dual-axis single-beam magnetometer to generate a z-axis control signal for a z-axis compensation coil oriented along the z-axis (step 301). The operations further comprise processing the photodetector signal to generate a y-axis control signal for a y-axis compensation coil oriented along the y-axis (step 302). The operations further comprise modifying a y-axis current to drive the y-axis compensation coil based on the y-axis control signal and a modulation pattern (step 303). The operations further comprise delivering the modified y-axis current to the y-axis compensation coil to mitigate background magnetic field components along the y-axis (step 304). The operations further comprise utilizing lock-in detection to estimate magnetic field components along the x-axis based on the z-axis control signal and the modulation pattern (step 305).

Referring back to FIG. 2, magnetic field detection system 101 includes a brief example of process 300 as implemented by the various components of system 101. In some examples, sensor mount 111 is placed on target 151 to contact magnetometer 121 with the surface of target 151. Controller 141 delivers current and activation signaling to magnetometer 121 to activate and initialize (e.g., calibrate, set temperature, etc.) magnetometer 121. Once activated and initialized, controller 141 delivers current and control signaling that drives magnetometer 121 to sense the target magnetic field generated by target 151. Coils 204-206 generate nulling magnetic fields oriented in the x, y, and z-axes to null background magnetic fields. Laser 201 emits a beam that passes through vapor cell 202. Photodetector 203 senses the beam after it passes through vapor cell 202. Photodetector 203 generates a signal that characterizes the absorbed beam and transfers the signal to the processing circuitry in controller 141.

The processing circuitry implements a control loop utilizing a first modulation pattern (referred to as b_{mod1}(t)) and a second modulation pattern (referred to as b_{mod2}(t)) to tune the fields produced by coils 204-206 to mitigate the background magnetic field and to read out magnetic fields detected by magnetometers 121 along the three orthogonal measurement axes. For example, the fields produced by coils 204-206 may not fully mitigate the background magnetic fields when magnetometer 121 is first turned on or the background field may change during the operation of magnetometer 121. The first modulation pattern comprises the form:

$${b}_{mod 1} ( t ) = {A}_{z 1} cos ( {ω}_{z 1} t ) \hat{z} + {A}_{y 1} sin ( {ω}_{y 1} t ) \hat{y}$$

where A_{z1} is the amplitude along the z-axis, A_{y1} it the amplitude along the y-axis, and ω_{z1} and ω_{y1} are angular frequencies. The amplitudes A_{z1} and A_{y1} are non-zero and typically range from 20 nT-200 nT. The angular frequencies ω_{z1} and ω_{y1} typically comprise 2πxx1000 Hz. The z-axis waveform of b_{mod1}(t) is cos(ω_{z1}t) while the y-axis waveform of b_{mod1}(t) is sin(coyjt). In some examples the angular frequencies, ω_{z1} and ω_{y1}, may differ between the y-axis component and the z-axis component of the modulation pattern b_{mod1}(t). The second modulation pattern comprises the form:

b_{mod2}(t)=A_{y2} cos(ω_{y2}t)ŷ
where A_{y2} is the amplitude and ω_{y2} is angular frequency. The amplitude A_{y2} is non-zero and typically ranges from 0.2-20 nT. The angular frequency ω_{y2} typically comprises 2π×250 Hz. These values are exemplary and may differ in other examples. The waveform cos(ω_{y2}t) of b_{mod2} (t) is aligned along the y-axis. The angular frequencies ω_{z1}, ω_{y1} and ω_{y2} may be equal or different. When these modulation patterns are applied by controller 141, changes in the transmitted light are proportional to detected magnetic fields according to:

$${S}_{1} ∝ ( {B}_{z} + {B}_{x} {A}_{y 2} cos ( {ω}_{y 2} t ) ) \times cos ( {ω}_{z 1} t ) + {B}_{y} sin ( {ω}_{y 1} t )$$

where Bₓ, B_{y}, B_{z} are the sum of components of the detected magnetic field along the x-axis, y-axis, and z-axis respectively.

The processing circuitry processes the photodetector signal received from magnetometer 121 using lock-in detection to generate a control signal for z-axis coils 206 (step 301). Lock-in detection of the photodetector signal for the z-axis is referenced with respect to the z-axis waveform of the first modulation pattern b_{mod1}(t). The processing circuitry modifies the current to drive z-axis coils 206 based on the z-axis control signal and the z-axis waveform of b_{mod1}(t). For example, the control output may modify the amplitude, phase, frequency content, or some other aspect of the current supplied to z-axis coils 206 to modify the magnetic field generated by z-axis coil 206. The processing circuitry delivers the modified current to z-axis coils 206 to mitigate background magnetic field along the z-axis. The processing circuitry then estimates the magnetic field components of the z-axis based on the control output for z-axis coils 206.

The processing circuitry processes the photodetector signal received from magnetometer 121 using lock-in detection to generate a control signal for y-axis coils 205 (step 302). Lock-in detection of the photodetector signal for the y-axis is referenced with respect to the y-axis waveform of the first modulation pattern b_{mod1}(t). For example, the control output may modify the amplitude, phase, frequency content, or some other aspect of the current supplied to y-axis coils 205 to modify the magnetic field generated by y-axis coil 205. The processing circuitry modifies the current to drive y-axis coils 205 based on the y-axis control signal, the y-axis waveform sin(ω_{y1}t) of the first modulation pattern b_{mod1}(t), and the y-axis waveform cos(ω_{y2}t) of the second modulation pattern b_{mod2}(t) (step 303). The processing circuitry delivers the modified current to y-axis coils 205 to mitigate background magnetic field along the y-axis (step 304). The processing circuitry estimates the magnetic field component along to the y-axis based on the control output for y-axis coils 205.

The processing circuitry processes the z-axis control signal and the second modulation pattern b_{mod2} (t) using lock-in detection to generate a control output for x-axis coils 204. Lock-in detection of the z-axis control signal is referenced with respect to the y-axis waveform cos(ω_{y2}t) of the second modulation pattern b_{mod2}(t). The processing circuitry modifies the current supplied to x-axis coils 204 based on the x-axis control output. The processing circuitry delivers the modified current to x-axis coils 204 to mitigate background magnetic fields along the propagation axis of the magnetometer light beam. The processing circuitry estimates the magnetic field component parallel to the x-axis based on the control output for x-axis coils 204 (step 305). For example, the processing circuitry may read out the magnetic field components along the x-axis based on the control output and the relationship given by S₁.

FIG. 4 illustrates process 400. Process 400 comprises an exemplary operation of magnetic field detection system 101 to sense magnetic fields along x, y, and z measurement axes of an intrinsically single-axis single-beam magnetometer where the x-axis is parallel to the propagation direction of the magnetometer light beam while the y and z axes are orthogonal to the light beam and to each other. Process 400 comprises an example of process 300 illustrated in FIG. 3, however process 300 may differ. In other examples, process 400 may differ. The operations of process 400 comprise processing a photodetector signal from the intrinsically single-axis single-beam magnetometer to generate a z-axis control signal for a z-axis compensation coil oriented along the z-axis (step 401). The operations further comprise modifying a y-axis current to drive a y-axis compensation coil oriented along the y-axis using a modulation pattern (step 402). The operations further comprise delivering the modified y-axis current to the y-axis compensation coil to mitigate background magnetic field components along the y-axis (step 403). The operations further comprise modifying an x-axis current to drive an x-axis compensation coil oriented along the x-axis using the modulation pattern (step 404). The operations further comprise delivering the modified x-axis current to the x-axis compensation coil to mitigate background magnetic field components along the x-axis (step 405). The operations further comprise utilizing lock-in detection to estimate magnetic field components along the y-axis based on the z-axis control signal and the modulation pattern (step 406). The operations further comprise utilizing lock-in detection to estimate magnetic field components along the x-axis based on the z-axis control signal and the modulation pattern (step 407).

Referring back to FIG. 2, magnetic field detection system 101 includes a brief example of process 400 as implemented by the various components of system 101. In some examples, sensor mount 111 is placed on target 151 to contact magnetometer 121 with the surface of target 151. Controller 141 delivers current and activation signaling to magnetometer 121 to activate and initialize magnetometer 121. Controller 141 delivers current and control signaling to magnetometer 121 that drives magnetometer 121 to sense the target magnetic field. Coils 204-206 generate nulling magnetic fields oriented in the x, y, and z-axes to block background magnetic field noise. Laser 201 emits a beam that passes through vapor cell 202. Photodetector 203 senses the beam after it passes through vapor cell 202. Photodetector 203 generates a signal that characterizes the absorbed beam and transfers the signal to the processing circuitry in controller 141.

The processing circuitry implements a control loop utilizing a first modulation pattern (referred to as b_{mod3}(t)) and a second modulation pattern (referred to as b_{mod4}(t)) to tune the fields produced by coils 204-206 to mitigate the background magnetic field and to read out detected magnetic fields along the three orthogonal axes. The first modulation pattern comprises the form:

b_{mod3}(t)=A_{z3} cos(ω_{z3}t)\hat{z}
where A_{z3} is the amplitude along the z-axis and ω_{z3} is the angular frequency. The amplitude A_{z3} is non-zero and typically ranges from 20 nT-200 nT. The angular frequency ω_{z3} typically comprises 2π×1000 Hz. The waveform cos(ω_{z3}t) of b_{mod3}(t) is aligned with the z-axis. The second modulation pattern comprises the form:

$${b}_{mod 4} ( t ) = {A}_{y 4} cos ( {ω}_{y 4} t ) \hat{y} + {A}_{x 4} sin ( {ω}_{x 4} t ) \hat{x}$$

where A_{y4} and Aₓ₄ are the amplitudes and ω_{y4} and ωₓ₄ are angular frequencies. The amplitudes A_{y4} and Aₓ₄ are non-zero and typically range from 0.2-20 nT. The angular frequencies ω_{y4} and ωₓ₄ typically range between 2π×1 Hz and 2π×500 Hz. These values are exemplary and may differ in other examples. The y-axis waveform of b_{mod4}(t) is cos(ω_{y4}t) while the x-axis waveform is sin(ωₓ₄t). The angular frequencies ω_{z3}, ω_{y4}, and ωₓ₄ may be equal or different. When these modulation patterns are applied by controller 141, changes in the transmitted light are proportional to detected magnetic fields according to:

$${S}_{1} ∝ ( {B}_{z} + {B}_{x} {A}_{y 4} cos ( {ω}_{y 4} t ) + {B}_{y} {A}_{x 4} sin ( {ω}_{x 4} t ) ) \times cos ( {ω}_{z 3} t )$$

where Bₓ, B_{y}, B_{z} are the sum of components of the detected magnetic field along the x-axis, y-axis, and z-axis respectively.

The processing circuitry processes the photodetector signal received from magnetometer 121 using lock-in detection to generate a control signal for z-axis coils 206 (step 401). Lock-in detection of the photodetector signal for the z-axis is referenced with respect to the z-axis waveform of the first modulation pattern b_{mod3}(t). The processing circuitry modifies the current to drive z-axis coils 206 based on the z-axis control signal and the z-axis waveform of b_{mod3} (t). The processing circuitry delivers the modified current to z-axis coils 206 to mitigate background magnetic field along the z-axis. The processing circuitry then estimates the magnetic field components of the z-axis based on the control output for z-axis coils 206.

The processing circuitry processes the z-axis control signal and the second modulation pattern b_{mod4}(t) using lock-in detection to generate a control signal for y-axis coils 205. For the y-axis, lock-in detection of the z-axis control signal is referenced with respect to the x-axis waveform sin(ωₓ₄t) of the second modulation pattern b_{mod4}(t). The processing circuitry modifies the current to drive y-axis coils 205 based on the y-axis control signal and the y-axis waveform cos(ω_{y4}t) of the second modulation pattern b_{mod4}(t) (step 402). The processing circuitry delivers the modified current to y-axis coils 205 to mitigate background magnetic field along the y-axis (step 403).

The processing circuitry processes the z-axis control signal and the second modulation pattern b_{mod4}(t) using lock-in detection to generate a control output for x-axis coils 204. For the x-axis, lock-in detection of the z-axis control signal is referenced with respect to the y-axis waveform cos(ω_{y4}t) of the second modulation pattern b_{mod4}(t). The processing circuitry modifies the current to drive x-axis coils 204 based on the x-axis control signal and the x-axis waveform sin(ωₓ₄t) of the second modulation pattern b_{mod4}(t) (step 404). The processing circuitry delivers the modified current to x-axis coils 204 to mitigate background magnetic field along the x-axis (step 405). The processing circuitry estimates the magnetic field components along the y-axis based on the control output for y-axis coils 205 (step 406). The processing circuitry estimates the magnetic field components along the x-axis based on the control output for x-axis coils 204 (step 407). For example, the processing circuitry may read out the magnetic field components along the x-axis and the y-axis based on the control outputs and the relationship given by S₂.

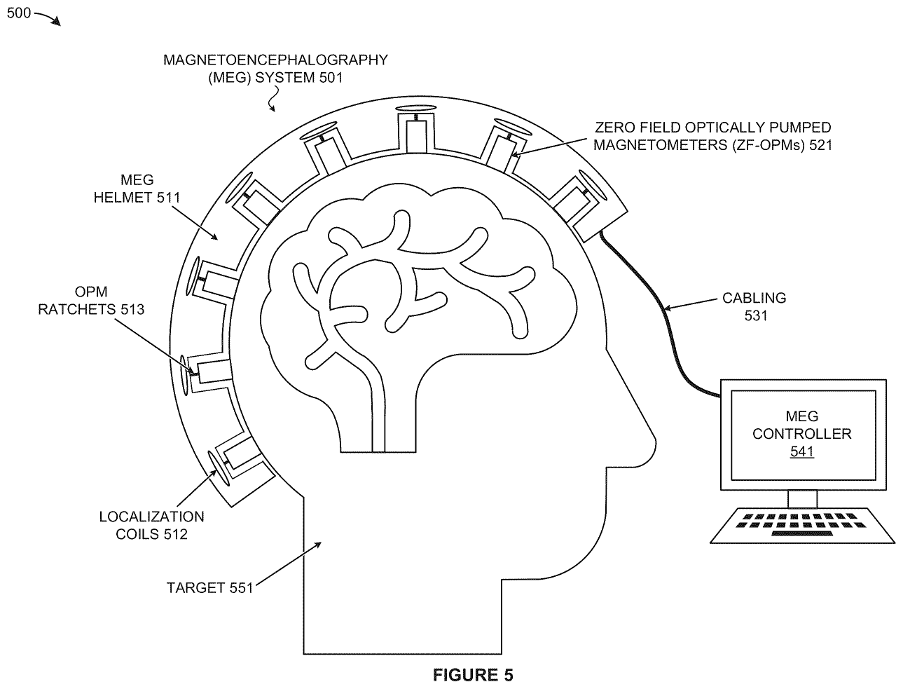

FIG. 5 comprises view 500. View 500 illustrates MEG system 501. MEG system 501 is an example of magnetic field detection system 101 illustrated in FIGS. 1 and 2, however system 101 may differ. MEG system 501 performs operations like detecting magnetic fields and relating the detected magnetic fields to neuronal activity for use in medical applications. Exemplary medical applications include identifying brain activity and diagnosing medical conditions like stroke, epilepsy, neuronal injuries, neuronal disorders, and/or other types of medical conditions relating to brain/neuron activity. MEG system 501 comprises MEG helmet 511, localization coils 512, OPM ratchets 513, ZF-OPMs 521, cabling 531, MEG controller 541, and target 551. In other examples, MEG system 501 may differ. In other examples, MEG system 501 may include fewer or additional components than those illustrated in FIG. 5. Likewise, the illustrated components of system 501 may include fewer or additional components, assets, or connections than shown.

In some examples, MEG helmet 511 is placed on target 551. Neuronal activity in target 551 generates a target magnetic field. MEG controller 541 supplies current and control signaling to ZF-OPMs 521 that drive ZF-OPMs 521 to measure the target magnetic field. ZF-OPMs 521 are representative of single beam ZF-OPMs intrinsically sensitive to one or two measurement axes. In the case of two-axis sensitivity, ZF-OPMs 521 are referred to as dual-axis single-beam ZF-OPMs. In the case of one-axis sensitivity, ZF-OPMs 521 are referred to as single-axis single-beam ZF-OPMs. The sensitivity axis or axes is determined by a first modulation pattern applied to the magnetometer by MEG controller 541. To enable three axis sensitivity, MEG controller 541 applies an additional modulation pattern to the current supplied to the compensation coils within ZF-OPMs 521. The additional modulation pattern modifies the magnetic fields produced by the compensation coils and allows MEG controller 541 to estimate vector components of the target magnetic field along the non-sensing axis (or axes) of ZF-OPMs 521 based on the signals received from ZF-OPMs 521.

Helmet 511 comprises a conformal MEG apparatus. Helmet 511 is shaped to conform to the geometry of a human head. Helmet 511 is wearable by target 551 and positions ZF-OPMs 521 in contact with the scalp of target 551. For example, helmet 511 may securely adhere ZF-OPMs 521 to the scalp of target 551 using mechanical constraints. Helmet 511 may be constructed from rigid plastic, carbon fiber, polymer, or other types of materials that provide structural support to helmet 511 and that do not interfere in the magnetic sensing operations of ZF-OPMs 521. Helmet 511 comprises slots that form channels to control the position and orientation of ZF-OPMs 521. For example, the slots may be shaped to constrain the three orientational degrees of freedom for each of ZF-OPMs 521 and two of the three locational degrees of freedom for each of ZF-OPMs 521 allowing for each of ZF-OPMs 521 to move along a single axis of motion in a single orientation. Helmet 511 may comprise support elements like padding, straps, cushions, and/or some other type of support system to support and position the head of target 551 within MEG helmet 511.

OPM ratchets 513 attach ZF-OPMs 521 to helmet 511. The couplings control one or more degrees of freedom in the position and orientation of ZF-OPMs 521. In this example, OPM ratchets 513 comprise ratchet mechanisms to contour ZF-OPMs 521 to the scalp of target 551, however in other examples, OPM ratchets 513 may instead comprise set screws, springs, pistons, pneumatics, clamps, and the like. As stated above, the slots are shaped to constrain the three orientational degrees of freedom and two of the three locational degrees of freedom for each of ZF-OPMs 521. OPM ratchets 513 control the last locational degree of freedom for each of ZF-OPMs 521. When helmet 511 is worn by target 551, OPM ratchets 513 propel ZF-OPMs 521 through their respective slots to contact target 551. For example, ratchets 513 may be tightened to move ZF-OPMs 521 towards target 551 and may be loosened to move ZF-OPMs 521 away from target 551. Once in contact with target 551, OPM ratchets 513 lock to secure the position of ZF-OPMs 521. Once locked, all six of the orientational and locational degrees of freedom for ZF-OPMs 521 are fixed.

Helmet 511 mounts localization coils 512. Coils 512 comprise loops of metallic wiring that generate an electromagnetic field in response to receiving electric current. For example, coils 512 may comprise copper or aluminum wiring. Coils 512 may comprise single or multiple loops of any shape and size. Coils 512 are embedded into the surface of helmet 511. Individual ones of coils 512 correspond to individual ones of ZF-OPMs 521 on a one-to-one basis. When powered, coils 512 generate magnetic waves that form coil magnetic fields. ZF-OPMs 521 measure the coil magnetic fields and report the field strength to controller 541. Controller 541 determines the location of ZF-OPMs 521 based on the reported field strengths, the orientational and locational constraints, and the locations of coils 512.

ZF-OPMs 521 comprise sensors to sense magnetic fields generated by brain activity of target 551 for MEG imaging. ZF-OPMs 521 also sense magnetic fields generated by coils 512 during localization. ZF-OPMs 521 are representative of either dual-axis single-beam ZF-OPMs or single-axis single-beam ZF-OPMs. As such, ZF-OPMs 521 are not intrinsically sensitive to all three measurement axes (e.g., x, y, and z). ZF-OPMs 521 generate signals proportional to the vector components of the sensed magnetic fields. The neuronal activity in the brain of target 551 comprises intercellular electromagnetic signals. ZF-OPMs 521 sense the magnetic component of the electromagnetic signals to detect neuronal activity. ZF-OPMs 521 form a sensor array that is contoured to the head of target 551 by helmet 511. ZF-OPMs 521 are coupled to controller 541 over cabling 531. Cabling 531 comprises sheathed metallic wires. For example, ZF-OPMs 521 may transfer signaling that characterizes the sensed magnetic fields to controller 541 over cabling 531. In some examples, cabling 531 may be replaced with, or used in addition with, a wireless transceiver system (e.g., antennas) to transfer communications between controller 541 and ZF-OPMs 521 over a wireless networking protocol like Bluetooth.

MEG controller 541 is representative of one or more computing devices configured to drive the operation of ZF-OPMs 521 and coils 512, localize ZF-OPMs 521, and generate MEG images that depict the neuronal activity in target 551. The one or more computing devices comprise processors, memories, lock-in amplifiers, modulators, PID controllers, and transceivers that are connected over bus circuitry. The processors may comprise CPUs, GPUs, DSPs, ASICs, FPGAs, and the like. The lock-in amplifiers may comprise demodulators and low-pass filters. The memories may comprise RAM, HDD, SSD, NVMe SSD, and the like. The memory stores software like operating systems, control applications, localization applications, PID control applications, MEG applications, sensor data, and the like. The processors retrieve and execute the software from the memory to drive the operation of controller 541.

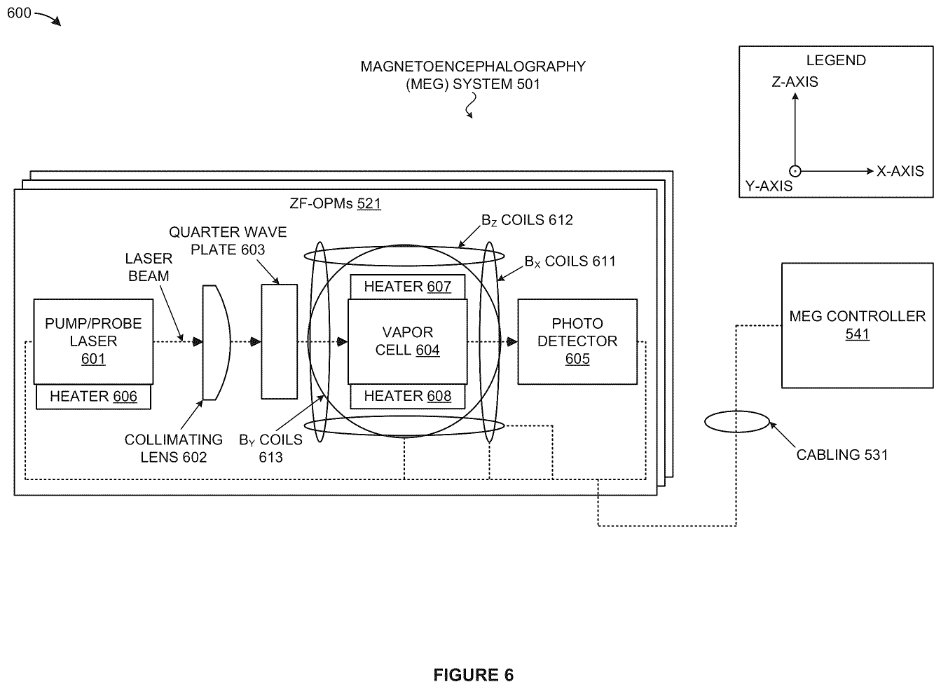

FIG. 6 comprises view 600. View 600 further illustrates ZF-OPMs 521. ZF-OPMs 521 comprises an example of magnetometers 121 illustrated in FIGS. 1 and 2, however magnetometers 121 may differ. ZF-OPMs 521 are referred to in the singular for sake of clarity. ZF-OPM 521 is representative of an intrinsically dual-axis single-beam magnetometer (e.g., sensitive to magnetic fields on both orthogonal axes with respect to the light beam propagation axis) or an intrinsically single-axis single-beam magnetometer (e.g., sensitive to magnetic fields on one orthogonal axis with respect to the light beam propagation axis). The single-beam design simplifies the ZF-OPM 521 as it requires fewer optical and hardware components and signal processing resources to operate when compared to conventional three-axes ZF-OPMs. ZF-OPM 521 comprises pump/probe laser 601, collimating lens 602, quarter wave plate 603, vapor cell 604, photodetector 605, heaters 606-608, Bₓ-coils 611, B_{z}-coils 612, and B_{y}-coils 613.

Coils 611-613 comprise three pairs of compensating coils to null background magnetic noise along the x, y, and z-axes with respect to the propagation direction of the laser beam produced by laser 601. Coils 611-613 may additionally generate bias fields to influence the measurement operations of ZF-OPM 521. Coils 611-613 are positioned around vapor cell 604. Each coil pair is positioned on its respective axis. Bₓ-coils 611 are positioned on both sides of vapor cell 604 oriented along the x-axis, B_{z}-coils 612 are positioned on both sides of vapor cell 604 oriented along the z-axis, and B_{y}-coils 613 are positioned on both sides of vapor cell 604 oriented along the y-axis. As illustrated in FIG. 5, the x-axis runs along the horizontal axis of the page, the z-axis runs along the vertical axis of the page, and the y-axis runs through the page. The x-axis runs through vapor cell 604 and is parallel to propagation direction of the laser beam emitted by pump/probe laser 601. Coils 611-613 comprise loops of metallic wiring within ZF-OPM 521 that generate an electromagnetic field in response to receiving electric current. For example, coils 611-613 may comprise copper or aluminum wiring.

In some examples, pump/probe laser 601 comprises a light source to optically pump and probe the metallic vapor housed by cell 604. Pump/probe laser 601 produces a single laser beam. Collimating lens 602 comprises a glass piece that focuses the laser beam along a single propagation axis. Quarter wave plate 603 is an optical device that alters the polarization state of the laser beam. For example, quarter wave plate 603 may circularly polarize the laser beam. Vapor cell 604 comprises an atomic device the encloses metallic vapor. Vapor cell 604 is positioned within the magnetic field generated by neuronal activity in target 551. Vapor cell 604 may comprise a glass vapor cell, a silicon-glass vapor cell, and/or another type of vapor cell. The metallic vapor is selected for comprising properties relating to magnetic field sensitivity. Typically, the metallic vapor comprises an alkali metal vapor like rubidium vapor, cesium vapor, or potassium vapor. Vapor cell 604 may enclose buffer gases (e.g., nitrogen or helium) in addition to the metallic vapor. Photodetector 605 comprises a light sensor to absorb and measure the intensity of the beam emitted by pump/probe laser 601. When the probe beam passes through vapor cell 604, a portion of the beam is absorbed by the metallic vapor. The remaining portion of the beam exits cell 604 and is detected photodetector 605. The amount of light absorbed by the metallic vapor correlates to the components of the external magnetic field at the location of vapor cell 604. Heaters 606-608 comprise resistive heat elements operatively coupled to pump/probe laser 601 and vapor cell 604. Heater 606 heats pump/probe laser 601 to facilitate beam generation. Heaters 607 and 608 heat vapor cell 604 to heat the vapor and increase cell pressure to facilitate magnetic field sensing. Coils 611-613 comprise a set of coils arranged along the x, y, and z axes of vapor cell 604. Coils 611-613 emit a compensation magnetic field to null or otherwise counteract background magnetic fields and/or to orient the sensing direction of ZF-OPM 521 depending on the modulation pattern.

MEG controller 541 routes control signaling and/or current to laser 601, photodetector 605, and coils 611-613 over cabling 531 and bus circuitry within ZF-OPM 521. Photodetector 605 routes magnetic field measurement signals to MEG controller 541 over the bus circuitry of ZF-OPM 521 and cabling 531. MEG controller 541 also delivers current to heaters 606-608 however these power connections are omitted for clarity. Although an intrinsically single-axis or dual-axis sensing device, ZF-OPM 521 is sensitive to magnetic fields in the y-axis and z-axis orthogonal to the light beam propagation axis as well as to magnetic fields in the x-axis parallel to the light beam propagation axis. To enable the three-axis sensitivity, MEG controller 541 applies the additional modulation pattern (e.g., b_{mod2}(t) or b_{mod4}(t)) to the currents supplied to coils 611-613 which allows controller 541 to estimate magnetic field components on the otherwise “non-sensing” axis or axes of ZF-OPM 521 based on the photodetector signal received from ZF-OPM 521.

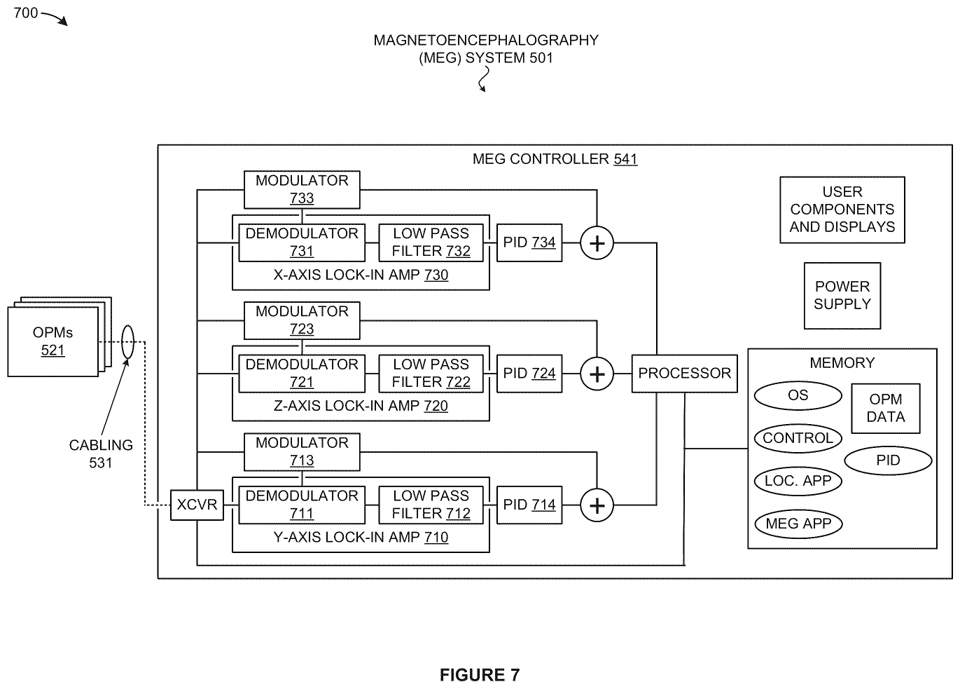

FIG. 7 comprises view 700. View 700 further illustrates MEG controller 541. MEG controller 541 comprises an example of controller 141 illustrated in FIGS. 1 and 2, however controller 141 may differ. MEG controller 541 comprises a transceiver (XCVR), y-axis lock-in amplifier (AMP) 710, z-axis lock in amplifier 720, x-axis lock in amplifier 730, modulators 713, 723, and 733, PID controllers 714, 724, and 734, a processor, memory, user components and display, and a power supply connected over bus circuitry. Y-axis lock-in amplifier 710 comprises demodulator 711 and low-pass filter 712. Z-axis lock-in amplifier 720 comprises demodulator 721 and low-pass filter 722. X-axis lock-in amplifier 730 comprises demodulator 731 and low-pass filter 732. Y-axis lock-in amplifier 710, modulator 713, and PID controller 714 correspond to B_{y}-coils 613 and the y-axis measurement axis. Z-axis lock-in amplifier 720, modulator 723, and PID controller 724 correspond to B_{z}-coils 612 and the z-axis measurement axis. X-axis lock-in amplifier 730, modulator 733, and PID controller 734 correspond to Bₓ-coils 611 and the x-axis measurement axis. The memory stores software like operating systems (OS), control applications (APP), localization applications, MEG applications, PID applications, and OPM data. The processor retrieves the software from the memory and executes the software to drive the operation of the MEG system 501 as described herein. The processor typically comprises an FPGA, however the FPGA may be replaced by, or used in addition to, a different processor type like CPU, ASIC, and the like.

The operating system manages the hardware and software resources of MEG controller 541. The control application controls the measurement operations of ZF-OPMs 521 and selects operating parameters (e.g., cell temperature) for ZF-OPMs 521. The localization application determines the spatial location of ZF-OPMs 521 based on the measured field strength of the magnetic field generated by localization coils 512, the spatial location of coils 512 within helmet 511, and the orientational/positional constrains applied to ZF-OPMs 521 by the helmet slots and OPM ratchets 513. The MEG application generates MEG images based on the measured magnetic field strength and the sensor location. The sensor data comprises operating parameters of ZF-OPM 521 like sensor Identifier (ID), slot ID, configuration parameters, and sensor performance metrics. The PID applications drive PID controllers 714, 724, and 734 to generate control outputs to tune the fields produced by coils 611-613. The control outputs may be used to read out the vector components of the target magnetic field along the x, y, and z-axes.

Lock-in amplifiers 710, 720, and 730 are used to derive the control signals for their respective compensation coils. Demodulators 711, 721, and 731 extract signals received from ZF-OPMs 521 for their respective compensation coils. Low-pass filters 712, 722, and 732 pass demodulated photodetector signals below a certain frequency to PID controllers 714, 724, and 734 and attenuate demodulated photodetector signals above the frequency. Modulators 713, 723, and 733 are used to generate components of the modulation patterns applied to the current/control signaling supplied to coils 611-613. Modulators 713, 723, and 733 are further used to generate the reference frequency and phase for lock-in amplifiers 710, 720, and 730 respectively. PID controllers 714, 724, and 734 process outputs from their respective low-pass filters to generate control signaling to tune the fields generates by coils 611-613. The processor modifies the current supplied to ZF-OPMs 521 using the control outputs supplied by PID controllers 714, 724, and 734 as well as the modulation patterns generated by modulators 713, 723, and 733 and delivers the modified currents to ZF-OPMS 521. The processor processes the control outputs generated by PID controllers 714, 724, and 734 (e.g., by executing the control application) to estimate magnetic field components along the x, y, and z-axes.

In some examples, MEG controller 541 may track the three components of the local magnetic field in a number of use cases. For example, MEG controller 541 may track faint orthogonal magnetic fields originated by an external source. MEG controller 541 may track both large and small changes in the local magnetic field simultaneously. If left uncompensated large fields can deteriorate OPM performance by saturating the OPM's dynamic range, inducing changes in the sensor gain, cross-axis talk, and noise among other effects, which inhibits the accurate tracking of small fields. The small changes can be originated by an external source, for instance by brain activity, while large changes can be artifacts caused by user motion in a static magnetic field. Some examples of these types of motion include head movement of target 551 along the x, y, and z-axes. As target 551's head moves in the presence of a static background field, the field at the location of the ZF-OPMs 521 changes accordingly. The background fields can be as large as a few nano-Tesla while the target magnetic field can be as small as tens of femto-Tesla. MEG controller 541 may track large changes in the magnetic field environment, for instance due to the opening of the shielding enclosure. MEG controller 541 may be used during the startup of ZF-OPMs 521 sensor to zero residual magnetic fields at the position of the sensor. MEG controller 541 may track the motion of target subject wearing ZF-OPMs 521.

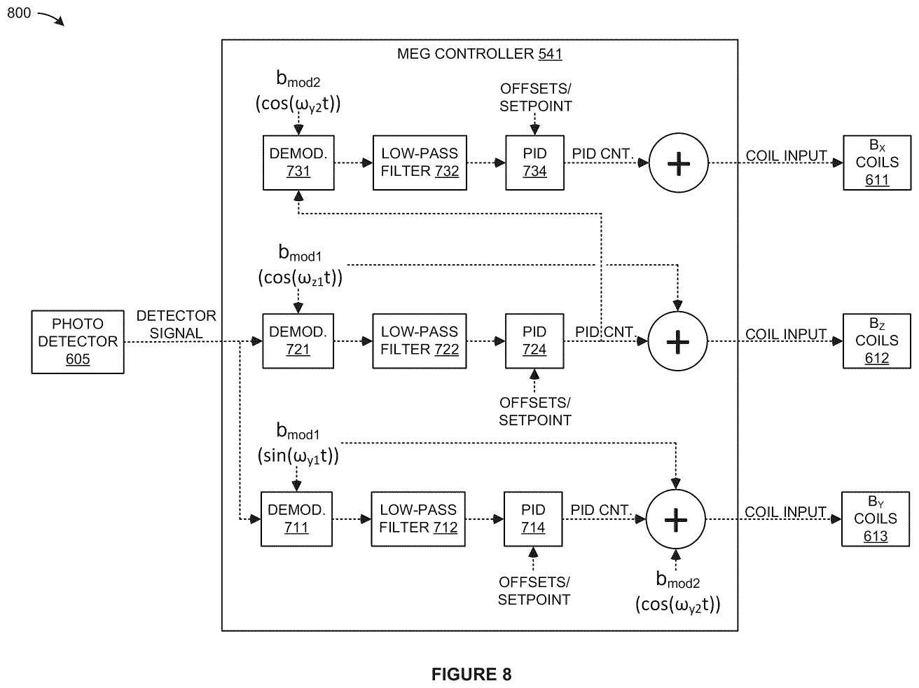

FIG. 8 comprises view 800. View 800 further illustrates MEG controller 541 to measure target magnetic fields along three-axes using an intrinsically dual-axis single-beam ZF-OPM (e.g., ZF-OPMs 521). In some examples, the processor of MEG controller 541 executes the PID applications to drive PID controllers 714, 724, and 734. Modulators 713 and 723 generate the waveforms sin(ω_{y1}t) and cos(ω_{z1}t), aligned with the y-axis and z-axis respectively. MEG controller 541 takes these waveforms and generates the first modulation pattern of the form:

$${b}_{mod 1} ( t ) = {A}_{z 1} cos ( {ω}_{z 1} t ) \hat{z} + {A}_{y 1} sin ( {ω}_{y 1} t ) \hat{y}$$

that is applied using coils 612 and 613. A_{z1} is the amplitude along the z-axis, A_{y1} is the amplitude along the y-axis, and ω_{z1} and ω_{y1} are the angular frequencies. The amplitudes A_{z1} and A_{y1} are non-zero and typically range from 20 nT-200 nT. The angular frequencies ω_{z1} and ω_{y1} typically comprise 2π×1000 Hz. Modulator 723 generates cos(ω_{z1}t) which is the z-axis waveform of b_{mod1}(t) while modulator 713 generates sin(ω_{y1}t) which is the y-axis waveform of b_{mod1} (t).

MEG controller 541 receives a photodetector signal from photodetector 605. MEG controller 541 processes the signal using z-axis lock in amplifier 720 to estimate magnetic fields along the z-axis. The signal is demodulated by demodulator 721 using the z-axis waveform cos(ω_{z1}t) of b_{mod1}(t) as a phase reference. The demodulated signal is then passed through low-pass filter 722 which attenuates frequencies above a threshold. The output of low-pass filter 722 is used by PID controller 724 as the error signal as well as additional information like offsets, setpoint, threshold, and the like. PID controller 724 processes these inputs to generate a control signal for the current driving B_{z}-coils 612 to cancel out the local magnetic field along the z-axis within the bandwidth of ZF-OPMs 521. In other examples, different controller types like model-predictive controllers may be used. The processor of MEG controller 541 adds the z-axis waveform cos(ω_{z1}t) of b_{mod1}(t) and the z-axis control signal generated by PID controller 724 to the electrical current driving B_{z}-coils 612 to cancel out the local magnetic field along the z-axis within the bandwidth of the sensor. The processor delivers the modified current to B_{z}-coils 612. The processor of MEG controller 541 executes the control application. The control application estimates the vector components of the magnetic field along the z-axis based on the control output of PID controller 724 and the relationship:

$${S}_{1} ∝ ( {B}_{z} + {B}_{x} {A}_{y 2} cos ( {ω}_{y 2} t ) \times cos ( {ω}_{z 1} t ) + {B}_{y} sin ( {ω}_{y 1} t )$$

where Bₓ, B_{y}, B_{z} are the sum of components of the detected magnetic field along the x-axis, y-axis, and z-axis respectively.

Contemporaneously, MEG controller 541 estimates magnetic fields along the y-axis using a similar approach. MEG controller 541 processes the photodetector signal using y-axis lock in amplifier 710. Demodulator 711 demodulates the photodetector signal using the y-axis waveform sin(ω_{y1}t) of b_{mod1}(t) generated by modulator 713 as a phase reference. The demodulated signal is passed to low-pass filter 712. Low-pass filter 712 attenuates energy above a cutoff frequency and passes the filtered signal to PID controller 714. The output of low-pass filter 712 is used by PID controller 714 as the error signal as well as additional information like offsets, setpoint, threshold, and the like. PID controller 714 processes these inputs to generate a y-axis control signal for B_{y}-coil 613 to cancel out the local magnetic field along the y-axis within the bandwidth of the sensor. The processor in MEG controller 541 adds the y-axis waveform sin(ω_{y1}t) of b_{mod1}(t) and the y-axis control signal generated by PID controller 714 to the electrical current driving B_{y}-coils 613 to cancel out the local magnetic field along the y-axis within the bandwidth of the sensor. The processor delivers the modified current to B_{y}-coils 613. The processor of MEG controller 541 executes the control application. The control application estimates the vector components of the magnetic field along the y-axis based on the y-axis control signal generated by PID controller 714 using the relationship given by S₁.

To achieve magnetic sensitivity along both orthogonal axes and the propagation axis of the light beam for an intrinsically dual-axis single-beam ZF-OPM, the processor of MEG controller 541 applies a second modulation pattern of the form:

b_{mod2}(t)=A_{y2} cos(ω_{y2}t)ŷ
to the current supplied to B_{y}-coils 613 in addition to the y-axis control signal and the y-axis waveform sin(ω_{y1}t) of b_{mod1}(t). The waveform cos(ω_{y2}t) of b_{mod2}(t) is generated by modulator 733, where the angular frequency ω_{y2} typically exceeds 2π×250 Hz to minimize interference with bio-magnetic signals of interest however the frequency may range from 2π×1 Hz to 2π×500 Hz. Magnetic field components along the propagation axis (x-axis) of the laser beam are then estimated using lock-in detection of the control signal generated by PID controller 724 for B_{z}-coils 612 using the waveform cos(ω_{y2}t) of b_{mod2}(t) as a phase reference. The z-axis control output of PID controller 724 is passed to demodulator 731. Demodulator 731 demodulates the signal and with the waveform cos(ω_{y2}t) of b_{mod2} (t) as a phase reference and passes the demodulated signal to low-pass filter 732. Low-pass filter 732 attenuates unwanted energy and passes the resulting signal to PID controller 734. PID controller 734 uses the output of low-pass filter 732 as the error signal and additional information like offsets, setpoint, threshold, and the like. PID controller 734 processes these inputs to generate a control signal to drive the current of the Bₓ-coils 611 to cancel out the local magnetic field along the x-axis within the bandwidth of the sensor. The processor of MEG controller 541 adds the x-axis control output generated by PID controller 734 to the electrical current driving the Bₓ-coils 611 to cancel out the local magnetic field along the x-axis within the bandwidth of the sensor. The processor delivers the modified current to Bₓ coils 611. The processor of MEG controller 541 executes the control application. The control application estimates the vector component of the magnetic field along the x-axis based on the x-axis control output of PID controller 734 using the relationship given by S₁.

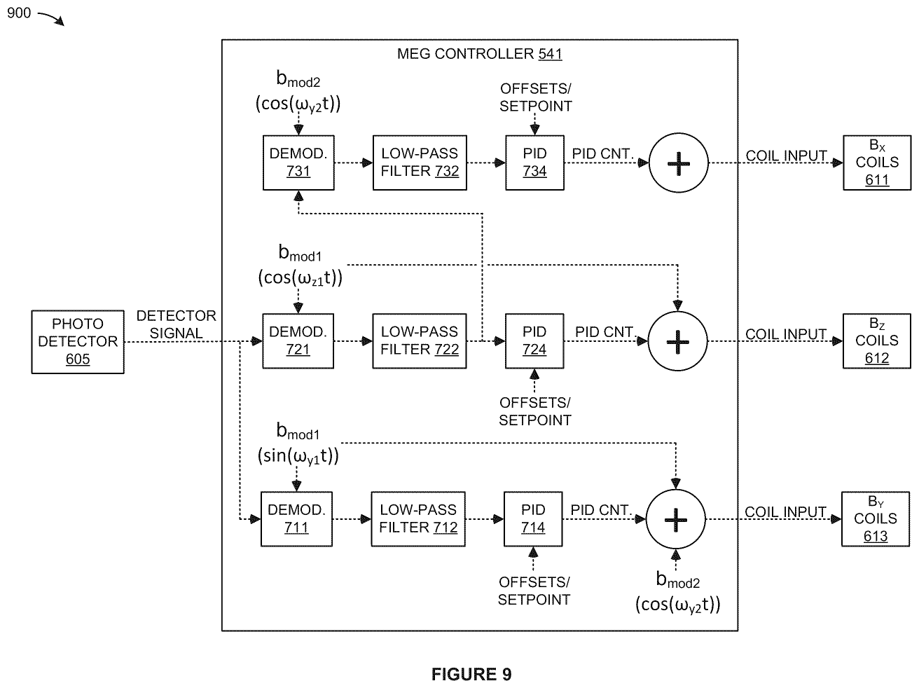

FIG. 9 comprises view 900. View 900 illustrates an alternative embodiment of MEG controller 541 to measure magnetic fields along three-axes using an intrinsically single-beam dual-axis ZF-OPM (e.g., ZF-OPM 521). In some examples, the output of low-pass filter 722 is used to derive an error signal for PID controller 734 to zero the local magnetic field along the x-axis instead of the control output generated by PID controller 724 as illustrated in FIG. 8. Lock in amplifier 730, PID controller 734, and the processor of MEG controller 541 process the output of low-pass filter 722 to zero the local magnetic field and estimate the vector-component of the magnetic field along the x-axis as described with respect to FIG. 8. Advantageously, this arrangement reduces the bandwidth and signaling load of PID controller 724.

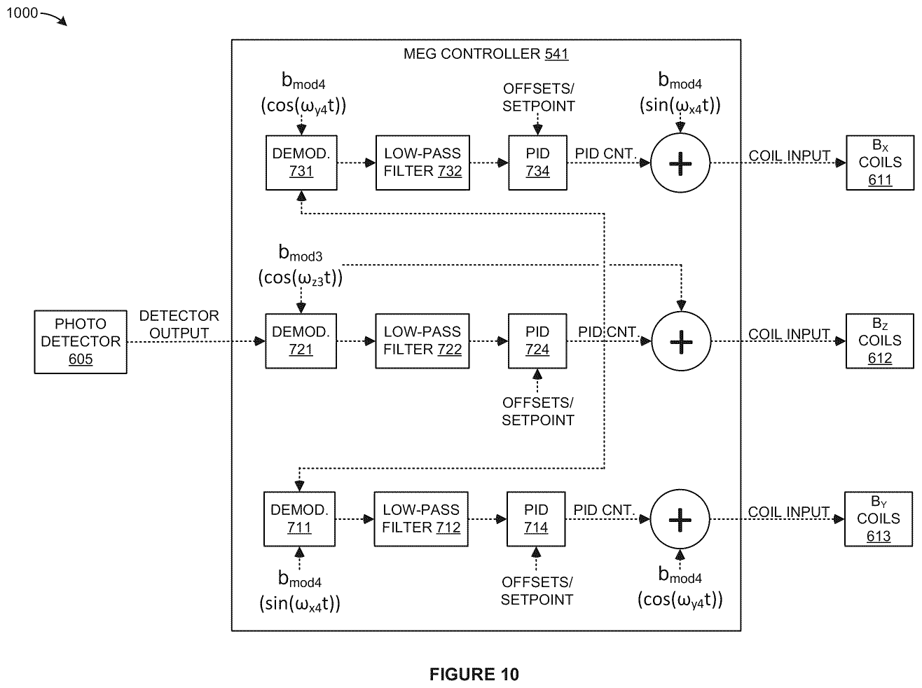

FIG. 10 comprises view 1000. View 1000 further illustrates MEG controller 541 to measure target magnetic fields along three-axes using an intrinsically single-axis single-beam ZF-OPM (e.g., ZF-OPMs 521). In some examples, the processor of MEG controller 541 executes the PID applications to drive PID controllers 714, 724, and 734. Modulator 723 generates the waveform cos(ω_{z3}t) aligned with the z-axis. MEG controller 541 takes this waveform and generates the first modulation pattern of the form:

b_{mod3}(t)=A_{z3} cos(ω_{z3}t)\hat{z}
which is applied using B_{z}-coils 612 where A_{z3} is the amplitude along the z-axis and ω_{z3} is the angular frequency. The amplitude A_{z3} is non-zero and typically ranges from 20 nT-200 nT. The angular frequency ω_{z3} typically comprises 2π×1000 Hz. MEG controller 541 receives a photodetector signal from photodetector 605. MEG controller 541 processes the signal using z-axis lock in amplifier 720 to estimate magnetic fields along the z-axis. Demodulator 721 demodulates the signal using the waveform cos(ω_{z3}t) of b_{mod3}(t) generated by modulator 723 as a phase reference. The demodulated signal is then passed through low-pass filter 722 which attenuates frequencies above a threshold. The output of low-pass filter 722 is used by PID controller 724 as the error signal as well as additional information like offsets, setpoint, threshold, and the like. PID controller 724 processes these inputs to generate a control signal for the current driving the B_{z}-coils 612 (PID CNT) to cancel out the local magnetic field along the z-axis within the bandwidth of the sensor. The processor of MEG controller 541 adds the z-axis control output generated by PID controller 724 as well as the waveform cos(ω_{z3} t) of b_{mod3}(t) to the current driving B_{z}-coils 612. The modified current is then supplied to B_{z}-coils 612. The processor of MEG controller 541 executes the control application which estimates the strength of the target magnetic field along the z-axis based on the output of PID controller 724 using the relationship:

$${S}_{2} ∝ ( {B}_{z} + {B}_{x} {A}_{y 4} cos ( {ω}_{y 4} t ) + {B}_{y} {A}_{x 4} sin ( {ω}_{x 4} t ) ) \times cos ( {ω}_{z 3} t )$$

where Bₓ, B_{y}, B_{z} are the sum of components of the detected magnetic field along the x-axis, y-axis, and z-axis respectively.

To achieve magnetic sensitivity along both orthogonal axes and the propagation axis of the light beam for an intrinsically single-axis single-beam ZF-OPM, MEG controller 541 applies a second modulation pattern of the form:

$${b}_{m o d 4} ( t ) = {A}_{y 4} cos ( {ω}_{y 4} t ) \hat{y} + {A}_{x 4} sin ( {ω}_{x 4} t ) \hat{x}$$

to B_{y}-coils 613 and Bₓ-coils 611 where A_{y4} and Aₓ₄ are the amplitudes and (ω_{y4} and ωₓ₄ are angular frequencies. The amplitudes A_{y4} and Aₓ₄ are non-zero and typically range from 0.2-20 nT. The angular frequencies ω_{y4} and ωₓ₄ typically range between 2π×1 Hz and 2π×500 Hz. Modulator 713 generates the y-axis waveform cos(ω_{y4}t) of b_{mod4}(t) while modulator 733 generates the x-axis waveform sin(ωₓ₄t) of b_{mod4}(t). The angular frequencies ω₃, ω_{y4}, ωₓ₄ and may be equal or different.

Magnetic field components along the x-axis are estimated using lock-in detection of the control output for B_{z}-coils 612 generated by PID controller 724 using the y-axis waveform cos(ω_{y4}t) of b_{mod4}(t) as a phase reference. The z-axis output of PID controller 724 is passed to demodulator 731. Demodulator 731 demodulates the signal with the y-axis waveform cos(ω_{y4}t) as phase reference and passes the demodulated signal to low-pass filter 732. Low-pass filter 732 attenuates unwanted energy and passes the resulting signal to PID controller 734. PID controller 734 uses the output of low-pass filter 732 as the error signal and additional information like offsets, setpoint, threshold, and the like. PID controller 734 processes these inputs to generate a control signal to drive the current of the Bₓ-coils 611 to cancel out the local magnetic field along the x-axis within the bandwidth of the sensor. The processor of MEG controller 541 adds the x-axis control output generated by PID controller 734 and the x-axis waveform sin(ωₓ₄t) of b_{mod4}(t) to the current driving Bₓ-coils 611. The processor delivers the modified current to Bₓ-coils 611. The processor executes the control application. The control application estimates the vector-component of the magnetic field along the x-axis based on the output of PID controller 734 using the relationship given by S₂.

Magnetic field components along the y-axis are estimated using lock-in detection of the control output for B_{z}-coils 612 generated by PID controller 724 using the x-axis waveform sin(ωₓ₄t) of b_{mod4}(t) as a phase reference. The z-axis output of PID controller 724 is passed to demodulator 711. Demodulator 711 demodulates the signal with the x-axis waveform sin(ωₓ₄t) as phase reference and passes the demodulated signal to low-pass filter 712. Low-pass filter 712 attenuates unwanted energy and passes the resulting signal to PID controller 714. PID controller 714 uses the output of low-pass filter 712 as the error signal and additional information like offsets, setpoint, threshold, and the like. PID controller 714 processes these inputs to generate a control signal to drive the current of the B_{y}-coils 613 to cancel out the local magnetic field along the y-axis within the bandwidth of the sensor. The processor of MEG controller 541 adds the y-axis control output generated by PID controller 714 and y-axis waveform cos(ω_{y4}t) of b_{mod4}(t) to the current driving B_{y}-coils 613. The processor supplies the modified current to Bₓ-coils 611. The processor of MEG controller 541 executes the control application. The control application estimates the vector-component of the magnetic field along the y-axis based on the output of PID controller 714 using the relationship given by S₂.

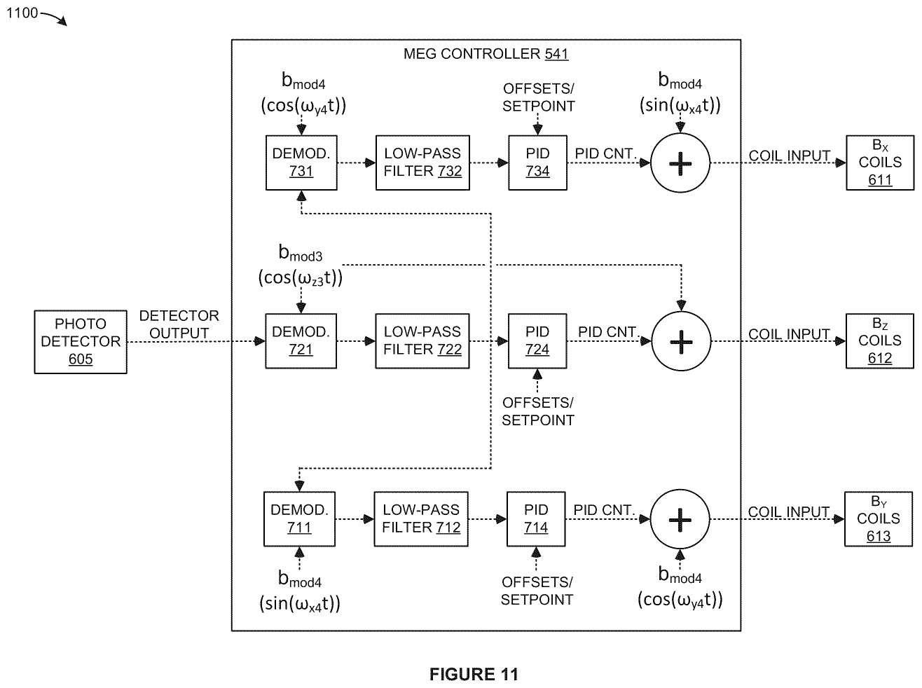

FIG. 11 comprises view 1100. View 1100 illustrates an alternative embodiment of MEG controller 541 to measure target magnetic fields along three-axes using an intrinsically single-beam single-axis ZF-OPM (e.g., ZF-OPM 521). In some examples, the output of low-pass filter 722 is used to derive error signals for PID controllers 714 and 734 to zero the local magnetic fields along the y-axis and x-axis, respectively, instead of the control output generated by PID controller 724 as illustrated in FIG. 10. Lock in amplifiers 710 and 730, PID controllers 714 and 734, and the processor process the output of low-pass filter 722 to zero the local magnetic field and estimate the vector-components of the magnetic field along the x-axis and y-axis as described with respect to FIG. 10. Advantageously, this arrangement reduces the bandwidth and signaling load of PID controller 724.

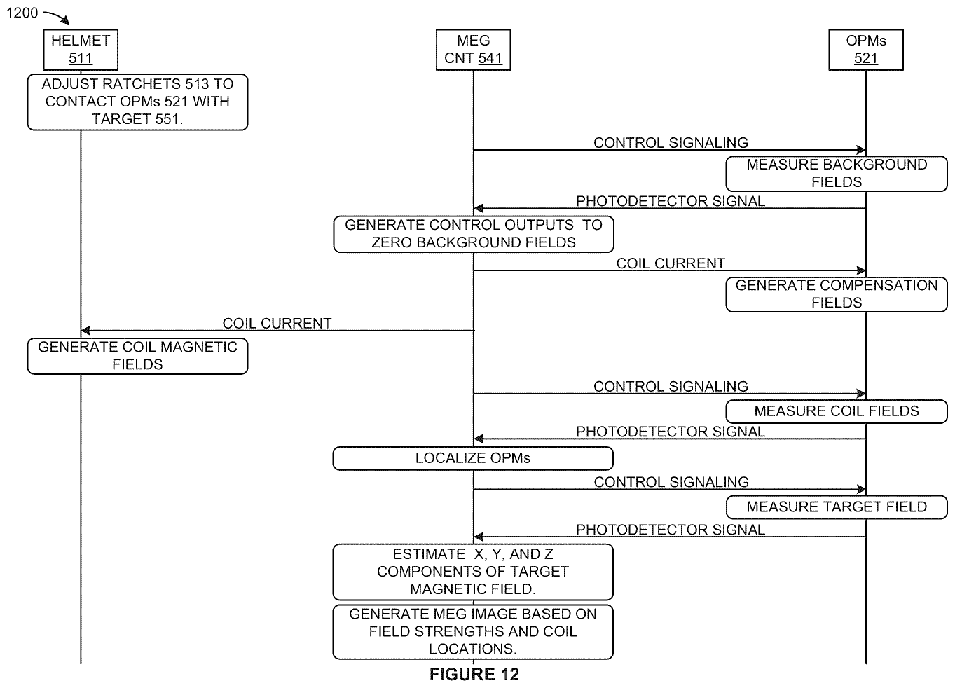

FIG. 12 illustrates process 1200. Process 1200 comprises an exemplary operation of MEG system 501 to measure target magnetic fields along three-axes using an intrinsically dual-axis single-beam ZF-OPM. In some examples, MEG helmet 511 is placed on the head of target 551. An operator adjusts OPM ratchets 513 to contact each of ZF-OPMs 521 with the scalp of target 551. The operator locks OPM ratchets 513 to secure the position and orientation of each of ZF-OPMs 521.

Once ZF-OPMs 521 are contoured to the surface of target 551, the operator initiates MEG imaging of target 551. MEG controller (MEG CNT) 541 receives user input and responsively executes the control application. The control application generates control signaling directing ZF-OPMs 521 to measure background magnetic field in the vicinity of target 551. MEG controller 541 transfers the control signaling and current to ZF-OPMs 521. ZF-OPMs 521 receive the control signaling and current. The compensation coils (e.g., Bₓ-coils 611, B_{z}-coils 612, and B_{y}-coils 613) generate compensation fields oriented along the x, y, and z-axes to mitigate the background magnetic field where the x-axis for each of ZF-OPMs 521 is oriented parallel to the propagation direction of their respective laser beams. The heaters (e.g., heaters 606-608) heat the vapor cells (e.g., vapor cell 604) and lasers (e.g., pump/probe laser 601) in ZF-OPMs 521. The vapor cells are positioned in the background magnetic field. The lasers emit a first pump beam that is circularly polarized at a resonant frequency of the vapor to polarize the atoms in the vapor cells. The beams pass through the collimating lenses (e.g., collimating lens 602) and quarter wave plates (e.g., quarter wave plate 603) and enter the vapor cells polarizing and probing the atoms. The presence of the background magnetic field alters the optical properties of the metallic vapor, which in turn are mapped onto the transmitted light beam. Changes in the optical properties correlate to the background magnetic field. The photodetectors (e.g., photodetector 605) detect the transmitted light beam by transducing changes in its optical properties into electronic signals. The photodetectors generate and transfer corresponding analog electronic signals that characterize the magnetic field to MEG controller 541 over cabling 531.

MEG controller 541 executes the PID applications to drive PID controllers 514, 524, and 534. MEG controller 541 receives the photodetector signals from ZF-OPMs 521. MEG controller 541 processes the signal using z-axis lock in amplifier 720. The output of lock in amplifier 720 is passed to PID controller 724 which generates a control signal to zero the background magnetic field along the z-axis for each of ZF-OPMs 521. MEG controller 541 applies the modulation pattern b_{mod1}(t) and adds the control signal to the current supplied to the B_{z}-coils 612 and delivers the current to the coils. Contemporaneously, MEG controller 541 processes the photodetector signal using y-axis lock in amplifier 710. The output of lock in amplifier 710 is passed to PID controller 714 which generates a control signal to zero the background magnetic field along the y-axis for each of ZF-OPMs 521. MEG controller 541 applies the modulation pattern b_{mod1}(t) and b_{mod2} (t) and adds the control signal to the current supplied to the B_{y}-coils 613 and delivers the current to the coils.

To zero the background fields along the x-axis, MEG controller 541 processes the output of PID controller 724 using lock-in amplifier 730 and the second modulation pattern b_{mod2} (t) as a phase reference. The output of lock in amplifier 730 is passed to PID controller 734 which generates a control signal to zero the background magnetic field along the x-axis for each of ZF-OPMs 521. MEG controller 541 adds the control signal to the current supplied to Bₓ-coils 611 and delivers the current to the coils. Bₓ-coils 611, B_{z}-coils 612, and B_{y}-coils 613 in ZF-OPMs 521 receive the respective currents from MEG controller 541 over cabling 531 and generate compensation fields. The compensation fields comprise a similar strength to the background magnetic field but are oriented in the opposite direction which nulls the background field. ZF-OPMs 521 and MEG controller 541 may repeat the above process (e.g., forming a control loop) to tune the compensation fields in response to changes in the background field and/or changes in orientation for ZF-OPMs 521 (e.g., by head movement of target 551).

Once the background field is nulled, the control application in MEG controller 541 initiates a localization process to determine the spatial locations of each of ZF-OPMs 521. The control application hosted by MEG controller 541 selects one of ZF-OPMs 521 for localization. The control application correlates the sensor ID of the selected OPM to a corresponding one of localization coils 512. MEG controller 541 supplies current to power the selected localization coil. The selected coil receives the current and generates a coil magnetic field. The magnitude of the coil magnetic field changes along the axis of motion (e.g., through the slot in helmet 511) of the selected OPM. The strength of the coil magnetic field decreases as the distance between the OPM and coil increases. Likewise, the strength of the coil magnetic field increases as the distance decreases. The control application transfers control signaling to selected OPM that directs the OPM to measure the strength of the coil magnetic field. The selected OPM measures the coil magnetic field as described above for measuring the background magnetic field and reports the field strength to MEG controller 541 over cabling 531.

MEG controller 541 receives the signals and processes the signals using lock in amplifiers 710, 720, and 730. The output of the lock in amplifiers is passed to PID controllers 714, 724, and 734 which generate control outputs. The control application determines the field strength of the coil magnetic field based on the control outputs. MEG controller 541 executes the localization application to determine the spatial location of the OPM. The localization application correlates the field strength reported by the OPM to a distance between the OPM and the coil. The localization application calculates the spatial location of the OPM based on the correlated distance, the known location of the coil on helmet 511, and the orientational and positional constraints of the OPM. The localization application stores the spatial location of the OPM in memory as sensor data. The localization application notifies the control application that localization of the selected OPM is complete. The control application drives MEG controller 541 to stop powering the coil and selects a new one of ZF-OPMs 521 for localization. MEG controller 541 repeats the localization process for the other ones of ZF-OPMs 521.

Once all of ZF-OPMs 521 on helmet 511 are located and their spatial locations are stored in the memory, the control application generates control signaling that directs ZF-OPMs 521 to measure the magnetic field generated by target 551. MEG controller 541 transfers the control signaling to ZF-OPMs 521. ZF-OPMs 521 measure the target magnetic field as described above for the background magnetic field and transfer the resulting photodetector signals to MEG controller 541.

MEG controller 541 receives the photodetector signals from ZF-OPMs 521. MEG controller 541 processes the signal using z-axis lock in amplifier 720. The output of lock in amplifier 720 is passed to PID controller 724 which generates a control output to determine target magnetic field strength along the z-axis for each of ZF-OPMs 521. Contemporaneously, MEG controller 541 processes the photodetector signal using y-axis lock in amplifier 710. The output of lock in amplifier 710 is passed to PID controller 714 which generates a control output to determine target magnetic field strength along the y-axis for each of ZF-OPMs 521. To determine the vector-component of the field along the x-axis for each of ZF-OPMs 521, MEG controller 541 applies the modulation pattern b_{mod2}(t) and processes the output of PID controller 724 using lock-in detector amplifier 730. The output of lock in amplifier 730 is passed to PID controller 734 which generates a control signal to determine the magnetic field along the x-axis for each of ZF-OPMs 521.

The control application processes the control outputs produced by PID controllers 714, 724, and 734 to determine the vector component of the target magnetic field along the y, z, and x-axes for each of ZF-OPM 521. The control application directs the MEG application to generate an MEG image depicting the neuronal activity in target 551. The MEG application generates an MEG image based on the detected target magnetic fields and the spatial locations of ZF-OPMs 521. MEG controller 541 displays the resulting MEG image on the user interface for review by the operator.

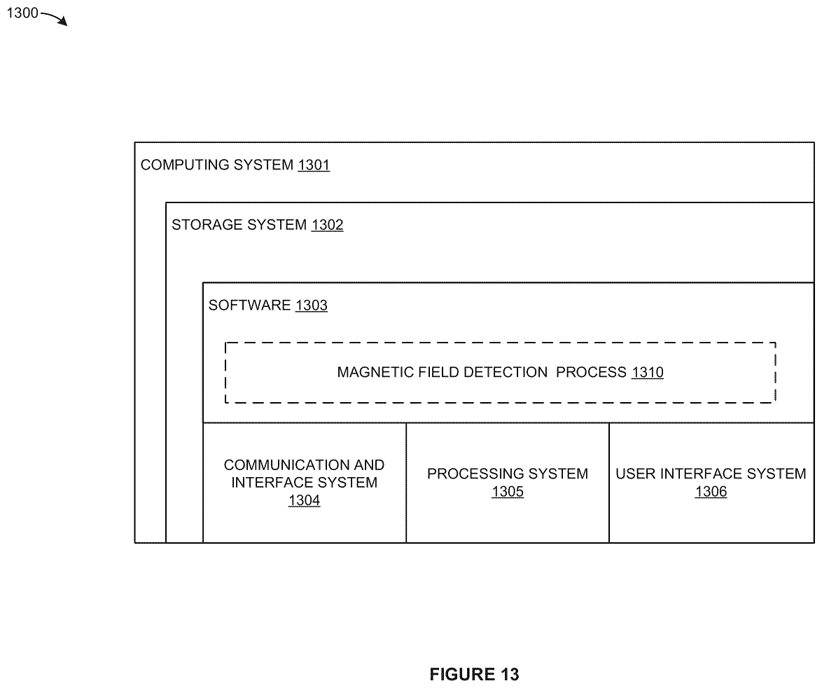

FIG. 13 comprises view 1300. View 1300 illustrates computing system 1301. Computing system 1301 is representative of any system or collection of systems with which the various operational architectures, processes, scenarios, and sequences disclosed herein for operating signal beam magnetometers to perform three-axis magnetic field detection. For example, computing system 1301 may be representative of magnetometers 121, controller 141, ZF-OPMs 521, MEG controller 541, and/or any other computing device contemplated herein. Computing system 1301 may be implemented as a single apparatus, system, or device or may be implemented in a distributed manner as multiple apparatuses, systems, or devices. Computing system 1301 includes, but is not limited to, storage system 1302, software 1303, communication interface system 1304, processing system 1305, and user interface system 1306. Processing system 1305 is operatively coupled with storage system 1302, communication interface system 1304, and user interface system 1306.

Processing system 1305 loads and executes software 1303 from storage system 1302. Software 1303 includes and implements magnetic field detection process 1310, which is representative of any of the three-axis magnetic field detection processes described with respect to the preceding Figures, including but not limited to the control operations for single and dual-axis single-beam magnetometers to sense magnetic fields along three orthogonal axes described with respect to the preceding Figures. For example, magnetic field detection process 1310 may be representative of the process 300 illustrated in FIG. 3, process 400 illustrated in FIG. 4, process 1200 illustrated in FIG. 12, and/or any other magnetic field detection process described herein. When executed by processing system 1305 to measure magnetic fields in three-axes using a single beam magnetometer, software 1303 directs processing system 1305 to operate as described herein for at least the various processes, operational scenarios, and sequences discussed in the foregoing implementations. Computing system 1301 may optionally include additional devices, features, or functionality not discussed for purposes of brevity.

Processing system 1305 may comprise a micro-processor and other circuitry that retrieves and executes software 1303 from storage system 1302. Processing system 1305 may be implemented within a single processing device but may also be distributed across multiple processing devices or sub-systems that cooperate in executing program instructions. Examples of processing system 1305 include general purpose CPUs, GPUs, DSPs, ASICs, FPGAs, analog computing devices, and logic devices, as well as any other type of processing device, combinations, or variations thereof.

Storage system 1302 may comprise any computer readable storage media readable by processing system 1305 and capable of storing software 1303. Storage system 1302 may include volatile, nonvolatile, removable, and/or non-removable media implemented in any method or technology for storage of information, such as computer readable instructions, data structures, program modules, or other data. Examples of storage media include RAM, read only memory, magnetic disks, optical disks, optical media, flash memory, virtual memory and non-virtual memory, magnetic cassettes, magnetic tape, magnetic disk storage or other magnetic storage devices, or any other suitable storage media. In no case is the computer readable storage media a propagated signal.

In addition to computer readable storage media, in some implementations storage system 1302 may also include computer readable communication media over which at least some of software 1303 may be communicated internally or externally. Storage system 1302 may be implemented as a single storage device but may also be implemented across multiple storage devices or sub-systems co-located or distributed relative to each other. Storage system 1302 may comprise additional elements, such as a controller, capable of communicating with processing system 1305 or possibly other systems.

Software 1303 (including magnetic field detection process 1310) may be implemented in program instructions and among other functions may, when executed by processing system 1305, direct processing system 1305 to operate as described with respect to the various operational scenarios, sequences, and processes illustrated herein. For example, software 1303 may include program instructions for measuring field components along the non-sensing axis of a dual-axis single beam ZF-OPM as described herein.

In particular, the program instructions may include various components or modules that cooperate or otherwise interact to carry out the various processes and operational scenarios described herein. The various components or modules may be embodied in compiled or interpreted instructions, or in some other variation or combination of instructions. The various components or modules may be executed in a synchronous or asynchronous manner, serially or in parallel, in a single threaded environment or multi-threaded, or in accordance with any other suitable execution paradigm, variation, or combination thereof. Software 1303 may include additional processes, programs, or components, such as operating system software, virtualization software, or other application software. Software 1303 may also comprise firmware or some other form of machine-readable processing instructions executable by processing system 1305.

In general, software 1303 may, when loaded into processing system 1305 and executed, transform a suitable apparatus, system, or device (of which computing system 1301 is representative) overall from a general-purpose computing system into a special-purpose computing system customized to measure magnetic fields along three-axes using single or dual-axis single-beam magnetometers as described herein. Indeed, encoding software 1303 on storage system 1302 may transform the physical structure of storage system 1302. The specific transformation of the physical structure may depend on various factors in different implementations of this description. Examples of such factors may include, but are not limited to, the technology used to implement the storage media of storage system 1302 and whether the computer-storage media are characterized as primary or secondary storage, as well as other factors.

For example, if the computer readable storage media are implemented as semiconductor-based memory, software 1303 may transform the physical state of the semiconductor memory when the program instructions are encoded therein, such as by transforming the state of transistors, capacitors, or other discrete circuit elements constituting the semiconductor memory. A similar transformation may occur with respect to magnetic or optical media. Other transformations of physical media are possible without departing from the scope of the present description, with the foregoing examples provided only to facilitate the present discussion.

Communication interface system 1304 may include communication connections and devices that allow for communication with other computing systems (not shown) over communication networks (not shown). Examples of connections and devices that together allow for inter-system communication may include network interface cards, antennas, power amplifiers, radiofrequency circuitry, transceivers, and other communication circuitry. The connections and devices may communicate over communication media to exchange communications with other computing systems or networks of systems, such as metal, glass, air, or any other suitable communication media. The aforementioned media, connections, and devices are well known and need not be discussed at length here.

Communication between computing system 1301 and other computing systems (not shown), may occur over a communication network or networks and in accordance with various communication protocols, combinations of protocols, or variations thereof. Examples include intranets, internets, the Internet, local area networks, wide area networks, wireless networks, wired networks, virtual networks, software defined networks, data center buses and backplanes, or any other type of network, combination of networks, or variation thereof. The aforementioned communication networks and protocols are well known and an extended discussion of them is omitted for the sake of brevity.

While some examples provided herein are described in the context of computing devices for magnetic field detection processes, it should be understood that the control and magnetometer systems and methods described herein are not limited to such embodiments and may apply to a variety of other environments and their associated systems. As will be appreciated by one skilled in the art, aspects of the present invention may be embodied as a system, method, computer program product, and other configurable systems. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

These and other changes can be made to the technology in light of the above Detailed Description. While the above description describes certain examples of the technology, and describes the best mode contemplated, no matter how detailed the above appears in text, the technology can be practiced in many ways. Details of the system may vary considerably in its specific implementation, while still being encompassed by the technology disclosed herein. As noted above, particular terminology used when describing certain features or aspects of the technology should not be taken to imply that the terminology is being redefined herein to be restricted to any specific characteristics, features, or aspects of the technology with which that terminology is associated. In general, the terms used in the following claims should not be construed to limit the technology to the specific examples disclosed in the specification, unless the above Detailed Description section explicitly defines such terms. Accordingly, the actual scope of the technology encompasses not only the disclosed examples, but also all equivalent ways of practicing or implementing the technology under the claims.

What is claimed is:

1. A method of operating a magnetic field detection system to sense magnetic fields along x, y, and z measurement axes of an intrinsically dual-axis single-beam magnetometer, the x-axis parallel to a propagation direction of a light beam of the magnetometer and the y and z axes orthogonal to the light beam and to each other, the method comprising:
processing a photodetector signal from the intrinsically dual-axis single-beam magnetometer to generate a z-axis control signal for a z-axis compensation coil oriented along the z-axis;
processing the photodetector signal to generate a y-axis control signal for a y-axis compensation coil oriented along the y-axis;
modifying a y-axis current to drive the y-axis compensation coil based on the y-axis control signal and a modulation pattern;
delivering the modified y-axis current to the y-axis compensation coil to mitigate background magnetic field components along the y-axis; and
utilizing lock-in detection to estimate magnetic field components along the x-axis based on the z-axis control signal and the modulation pattern.

2. The method of claim 1 further comprising:
modifying a z-axis current for the z-axis compensation coil based on the z-axis control signal; and
delivering the modified z-axis current to the z-axis compensation coil to mitigate background magnetic field components along the z-axis.

3. The method of claim 2 further comprising:
generating an x-axis control signal for an x-axis compensation coil oriented along the x-axis based on the z-axis control signal and the modulation pattern;
modifying an x-axis current for the x-axis compensation coil based on the x-axis control signal; and
delivering the modified x-axis current to the x-axis compensation coil to mitigate background magnetic field components along the x-axis.

4. The method of claim 3 wherein:
utilizing lock-in detection to estimate the magnetic field components along the x-axis based on the z-axis control signal and the modulation pattern comprises estimating the magnetic field components along the x-axis based on the x-axis control signal.

5. The method of claim 1 further comprising:
utilizing lock-in detection to estimate magnetic field components along the z-axis based on the z-axis control signal; and
utilizing lock-in detection to estimate magnetic field components along the y-axis based on the y-axis control signal.

6. The method of claim 1 wherein the modulation pattern comprises a sinusoidal magnetic field of a form A_{y2} cos(ω_{y2}t)ŷ where A_{y2} is amplitude and ω_{y2} is angular frequency.

7. The method of claim 1 wherein utilizing lock-in detection to estimate the magnetic field components along the x-axis comprises utilizing lock-in detection to estimate the magnetic field components along the x-axis based on the modulation pattern and an input to a controller that generates the z-axis control signal.

8. A method of operating a magnetic field detection system to sense magnetic fields along x, y, and z measurement axes of an intrinsically single-axis single-beam magnetometer, the x-axis parallel to a propagation direction of a light beam of the magnetometer and the y and z axes orthogonal to the light beam and to each other, the method comprising:
processing a photodetector signal from the intrinsically single-axis single-beam magnetometer to generate a z-axis control signal for a z-axis compensation coil oriented along the z-axis;
modifying a y-axis current to drive a y-axis compensation coil oriented along the y-axis using a modulation pattern;
delivering the modified y-axis current to the y-axis compensation coil to mitigate background magnetic components along the y-axis;
modifying an x-axis current to drive an x-axis compensation coil oriented along the x-axis using the modulation pattern;
delivering the modified x-axis current to the x-axis compensation coil to mitigate background magnetic field components along the x-axis;
utilizing lock-in detection to estimate magnetic field components along the y-axis based on the z-axis control signal and the modulation pattern; and
utilizing lock-in detection to estimate magnetic field components along the x-axis based on the z-axis control signal and the modulation pattern.

9. The method of claim 8 further comprising:
modifying a z-axis current for the z-axis compensation coil based on the z-axis control signal; and
delivering the modified z-axis current to the z-axis compensation coil to mitigate background magnetic field components along the z-axis.

10. The method of claim 9 further comprising:
generating a y-axis control signal for the y-axis compensation coil based on the z-axis control signal and the modulation pattern; and wherein:
modifying the y-axis current for the y-axis compensation coil comprises modifying the y-axis current for the y-axis compensation coil based on the y-axis control signal for and the modulation pattern; and
utilizing lock-in detection to estimate the magnetic field components along the y-axis comprises estimating the magnetic field components along the y-axis based on the y-axis control signal.

11. The method of claim 10 further comprising:
generating an x-axis control signal for the x-axis compensation coil based on the z-axis control signal and the modulation pattern; and wherein:
modifying the x-axis current for the x-axis compensation coil comprises modifying the x-axis current based on the x-axis control signal and the modulation pattern; and
utilizing lock-in detection to estimate the magnetic field components along the x-axis comprises estimating the magnetic field components along the x-axis based on the x-axis control signal.

12. The method of claim 8 further comprising utilizing lock-in detection to estimate magnetic field components along the z-axis based on the z-axis control signal.

13. The method of claim 8 wherein:
the modulation pattern comprises a sinusoidal magnetic field of a form A_{y4} cos(ω_{y4}t)ŷ+Aₓ₄ sin(ωₓ₄t)\hat{x} where A_{y4} is amplitude along the y-axis, Aₓ₄ is amplitude along the x-axis, and ωₓ₄ and ω_{y4} are angular frequencies.

14. The method of claim 8 wherein:
utilizing lock-in detection to estimate the magnetic field components along the y-axis based on the z-axis control signal and the modulation pattern comprises utilizing lock-in detection to estimate the magnetic field components along the y-axis based on the modulation pattern and an input to a controller that generates the z-axis control signal; and
utilizing lock-in detection to estimate the magnetic field components along the x-axis based on the z-axis control signal and the modulation pattern comprises utilizing lock-in detection to estimate magnetic field components along the x-axis based on the modulation pattern and the input to the controller that generates the z-axis control signal.

15. A magnetic field detection system to sense magnetic fields along x, y, and z measurement axes of an intrinsically dual-axis single-beam magnetometer, the x-axis parallel to a propagation direction of a light beam of the magnetometer and the y and z axes orthogonal to the light beam and to each other, the system comprising:
processing circuitry configured to:
process a photodetector signal from the intrinsically dual-axis single-beam magnetometer to generate a z-axis control signal for a z-axis compensation coil oriented along the z-axis;
process the photodetector signal to generate a y-axis control signal for a y-axis compensation coil oriented along the y-axis;
modify a y-axis current to drive the y-axis compensation coil based on the y-axis control signal and a modulation pattern;
deliver the modified y-axis current to the y-axis compensation coil to mitigate background magnetic field components along the y-axis; and
utilize lock-in detection to estimate magnetic field components along the x-axis based on the z-axis control signal and the modulation pattern.

16. The system of claim 15 wherein the processing circuitry is further configured to:
modify a z-axis current for the z-axis compensation coil based on the z-axis control signal; and
deliver the modified z-axis current to the z-axis compensation coil to mitigate background magnetic field components along the z-axis.

17. The system of claim 16 wherein the processing circuitry is further configured to:
generate an x-axis control signal for an x-axis compensation coil based on the z-axis control signal and the modulation pattern;
modify an x-axis current for the x-axis compensation coil based on the x-axis control signal; and
deliver the modified x-axis current to the x-axis compensation coil to mitigate background magnetic field components along the x-axis.

18. The system of claim 17 wherein the processing circuitry is configured to estimate the magnetic field components along the x-axis based on the x-axis control signal.

19. The system of claim 15 wherein the processing circuitry is further configured to:
utilize lock-in detection to estimate magnetic field components along the z-axis based on the z-axis control signal;
utilize lock-in detection to estimate magnetic field components along the y-axis based on the y-axis control signal; and
the modulation pattern comprises a sinusoidal magnetic field of a form A_{y2} cos(ω_{y2}t)ŷ where A_{y2} is amplitude and ω_{y2} angular frequency.

20. The system of claim 15 wherein the processing circuitry is to utilize lock-in detection to estimate the magnetic field components along the x-axis based on the modulation pattern and an input to a controller that generates the z-axis control signal.
